(19) United States
(12) Patent Application Publication
(10) Pub. No.: US 2024/0368700 A1
(43) Pub. Date: Nov. 7, 2024

(54) CANCER CLASSIFICATION AND PROGNOSIS BASED ON SILENT AND NON-SILENT MUTATIONS

(71) Applicant: RAMOT AT TEL-AVIV UNIVERSITY LTD., Tel Aviv (IL)

(72) Inventors: Tamir TULLER, Tel Aviv (IL); Tal GUTMAN, Tel Aviv (IL)

(21) Appl. No.: 18/562,317

(22) PCT Filed: May 19, 2022
(86) PCT No.: PCT/IL2022/050522

§ 371 Date: Nov. 19, 2023

Related U.S. Application Data
(60) Provisional application No. 63/190,712, filed on May 19, 2021.

(51) Int. Cl. C12Q 1/6886; G16B 20/00; G16B 40/20

(52) U.S. Cl. CPC C12Q 1/6886; G16B 20/00; G16B 40/20; C12Q 2600/118; C12Q 2600/156

(57) ABSTRACT

Methods of determining a type of cancer in a subject or estimating survival time after diagnosis of a subject comprising employing a machine learning model to evaluate mutations that are not exonic non-synonymous mutations are provided. Methods comprising training a machine learning model are also provided.

### CROSS REFERENCE TO RELATED APPLICATIONS

This application claims the benefit of priority of U.S. Provisional Patent Application No. 63/190,712 filed on May 19, 2021 titled “CANCER CLASSIFICATION AND PROGNOSIS BASED ON SILENT AND NON-SILENT MUTATIONS”, the contents of which are incorporated herein by reference in their entirety.

### FIELD OF INVENTION

The present invention is in the field of cancer diagnostics.

### BACKGROUND OF THE INVENTION

The rapid developments of New Generation Sequencing (NGS) technologies and acceleration of computational abilities over the past few years have led to the availability of extensive genomic information. Various research utilizing these high-dimensional data establish cancer as a group of highly heterogeneous genomic diseases, characterized by large inter-tumor and intra-tumor diversities. Moreover, common genetic features were repeatedly identified among patients of different cancer types and significant diversities were found among patients diagnosed with the same cancer type. These findings highlight the need for personalized, gene-targeted cancer treatments.

By now, hundreds of genes have been recognized as cancer drivers and many more are currently being researched. Some, like TP53, BRAF, EGFR or IDH1 have already been targeted for gene therapy. Nonetheless, there are still numerous obstacles to overcome in order to fully unravel the cancer genomic landscape. Currently, most contemporary research is based on data derived by Whole Exome Sequencing (WES). In addition, most studies focus exclusively or predominantly on non-silent mutations; alterations in the coding regions that cause a change in the amino-acid sequence of the produced protein. Silent mutations, such as modifications in introns, untranslated-regions (UTR′5 and UTR′3) or even synonymous mutations in the coding region itself are by and large excluded from the analyses.

Yet, cancerous silent mutations can have detrimental effects on gene expression, which in some cases can even lead to consequences more significant than non-silent mutations. Mutations in regulatory regions, such as promoters or enhancers, can destroy or form new transcription-factor binding sites and cause changes in transcriptional regulation. Mutations in the untranslated regions can affect translation regulation or modify microRNA binding sites and thus impact mRNA stability. Synonymous mutations can alter all aspects of gene expression, impacting translation rates, protein-folding, transcription, mRNA stability and splicing. Overall, silent mutations can modify all phases of the gene expression process, causing amplification or reduction in protein quantities. Hence, even though most silent mutations do not cause a change in protein functionality, they can dramatically change protein abundance and can therefore influence cancer fitness.

The incredible heterogeneity of cancerous genomes, even for patients who presumably possess the same cancer type, highly complicates predictive tasks. When examining only non-silent mutations one misses a large part of the complex mutational patterns of these cancerous genomes. Additionally, silent driver mutations, even though considered today as infrequent compared to non-silent drivers, could be highly influential and thus also beneficial for predictive models. Indeed, there are previous studies that have demonstrated that silent mutations or non-silent mutations that modulate gene expression can significantly affect the phenotype of the cancer cell and its survival.

Predictive models and analytical methods that integrate silent mutations and thus provided a broader understanding of the genomic landscape profoundly linked with cancer development and progression are greatly needed.

### SUMMARY OF THE INVENTION

The present invention provides methods of determining a type of cancer in a subject comprising employing a machine learning model to evaluate mutations that are not exonic non-synonymous mutations. Methods of estimating survival time after diagnosis of a subject are also provided. Methods comprising training a machine learning model are also provided.

According to a first aspect, there is provided a method of determining a type of cancer in a subject or estimating survival time after diagnosis of a subject, the method comprising:
a. receiving genomic mutation data from the cancer wherein the data comprises mutations that are not exonic non-synonymous mutations;
b. applying a trained machine learning (ML) model to the received genomic mutation data;
thereby determining a type of cancer in a subject or estimating survival time after diagnosis for the subject.

According to some embodiments, the data comprises mutations found in the cancer which are absent from healthy tissue of the subject.

According to some embodiments, the method is a method of determining cancer type and the ML model was trained on a training set comprising the genomic mutation data from cancer patients with known cancer types and the ML model outputs a classification of the cancer in the subject as one of the known cancer types.

According to some embodiments, the method is a method of estimating survival time after diagnosis of the subject and the ML model was trained on a training set comprising the genomic mutation data from cancer patients with known survival times from diagnosis and the ML model outputs an estimated survival time for the subject.

According to some embodiments, the training set comprises only mutations that appear in at least two of the cancer patients with known cancer types or known survival times from diagnosis.

According to some embodiments, the mutations are selected from: mutations in 3′ and 5′ untranslated regions (UTRs) of genes, mutations in introns of genes, mutations in regions flanking genes, and exonic synonymous mutations.

According to some embodiments, flanking regions comprise untranscribed sequences within 5 kb of a transcriptional start site of genes, within 5 kb of a transcriptional termination site of genes or both.

According to some embodiments, the genomic mutation data comprises:
a. all UTR mutations in deep sequencing data from the subject, the cancer patients or both;
b. all intronic mutations in deep sequencing data from the subject, the cancer patients or both;
c. all flanking region mutations in deep sequencing data from the subject, the cancer patients or both;
d. all synonymous exonic mutations in deep sequencing data from the subject, the cancer patients or both; or
e. a combination thereof.

According to some embodiments, the genomic mutation data further comprises exonic non-synonymous mutations.

According to some embodiments, the genomic mutation data comprises all exonic non-synonymous mutations in deep sequencing data from the subject, the cancer patients or both.

According to some embodiments, the deep sequencing is whole exome sequencing (WES).

According to some embodiments, the genomic mutation data comprises all mutations found in WES data from the subject, the cancer patients or both.

According to some embodiments, the cancer is selected from adrenal cancer, bladder cancer, urothelial cancer, breast cancer, cervical cancer, bile duct cancer, colon cancer, lymphoid cancer, esophageal cancer, brain cancer, head and neck cancer, renal cancer, liver cancer, lung cancer, mesodermal cancer, ovarian cancer, pancreatic cancer, endocrine cancer, neuroendocrine cancer, prostate cancer, rectal cancer, skin cancer, bone cancer, soft tissue cancer, stomach cancer, testicular cancer, thyroid cancer, uterine cancer and uveal cancer.

According to some embodiments, the genomic mutation data comprises intronic mutations and the cancer is selected from cervical cancer, colon cancer, brain cancer, renal cancer, and liver cancer.

According to some embodiments, the genomic mutation data comprises UTR mutations or flanking region mutations and the cancer is selected from cervical cancer, bone cancer and soft tissue cancer.

According to some embodiments, the genomic mutation data comprises UTR mutations, intronic mutations, flanking region mutations, exonic synonymous mutations and exonic non-synonymous mutations and the cancer is selected from bladder cancer, urothelial cancer, breast cancer, cervical cancer, colon cancer, renal cancer, liver cancer, lung cancer, ovarian cancer, bone cancer, soft tissue cancer, skin cancer, thyroid cancer and uterine cancer.

According to some embodiments, the genomic mutation data comprises UTR mutations, intronic mutations, flanking region mutations, exonic synonymous mutations and exonic non-synonymous mutations and the cancer is selected from breast cancer, colon cancer, brain cancer, renal cancer, liver cancer, ovarian cancer, bone cancer, soft tissue cancer, thyroid cancer and uterine cancer.

According to some embodiments, the genomic mutation data is from a cancer biopsy or liquid biopsy.

According to some embodiments, the method further comprises administering to the subject a therapeutic agent known to treat the determined cancer type.

According to some embodiments, the method further comprises administering an additional therapeutic treatment to a subject with an expected survival time below a predetermined threshold.

According to another aspect, there is provided a method comprising:
training a machine learning (ML) model to determine a type of cancer in a subject or estimate survival time after diagnosis of a subject, on a training set, the method comprising:
   i. receiving genomic data; and
   ii. extracting from the received genomic data mutations, wherein the mutations are not exonic non-synonymous mutations;
wherein the training set is generated by labeling the mutations as coming from a cancer of a specific type or from a subject that survived for a specific amount of time after diagnosis and combining a plurality of mutations and their labels together to form the training set, wherein the plurality comprises labels of cancers from at least two cancer types or labels from subjects that survived for different amounts of time.

According to some embodiments, the method further comprises at an inference step applying the trained ML model to genomic mutation data received from a cancer wherein the received genomic data comprises mutations that are not exonic non-synonymous mutations and outputting a determined type of cancer or an estimated survival time.

According to some embodiments, the inference step comprises a method of the invention.

According to another aspect, there is provided a method of evaluating a cancer, the method comprising receiving a sample comprising DNA from the cancer and detecting in the DNA a silent mutation in a gene selected from those provided in Table 5, thereby evaluating a cancer.

According to some embodiments, the cancer is selected from a cancer type provided in Table 5 and wherein the gene is selected from those whose mutation was observed in the cancer type.

According to some embodiments, the evaluating comprises determining a driver gene or driver mutation in the cancer.

According to some embodiments, the method further comprises administering to a subject that provided the sample an anticancer therapy that targets the determined driver gene, another gene is a biological pathway comprising the determined driver gene or the driver mutation.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D: TCGA data characteristics. Description of the data retrieved from TCGA after initial preprocessing (discarding patients with missing genomic or clinical data and patients with multiple genomic samples). Overall, 9,915 patients across 33 cancer types are included in the study. (1A) Bar chart of patient distribution across cancer types. ACC—Adrenocortical carcinoma, BLCA—Bladder Urothelial Carcinoma, BRCA—Breast Invasive Carcinoma, CESC—Cervical Squamous Cell Carcinoma, CHOL—Cholangiocarcinoma, COAD—Colon Adenocarcinoma, DLBC—Lymphoid Neoplasm Diffuse Large B-cell Lymphoma, ESCA—Esophageal Carcinoma, GBM—Glioblastoma, HNSC—Head and Neck Squamous Cell Carcinoma, KIRC—Kidney Renal Clear Cell Carcinoma, KIRP— kidney renal papillary cell carcinoma, LGG—Low Grade Gliomas, LIHC—Liver Hepatocellular Carcinoma, LUAD—Lung Adenocarcinoma, LUSC—Lung Squamous Cell Carcinoma, MESO—Mesothelioma, OV—Ovarian Serous Cystadenocarcinoma, PAAD—Pancreatic Adenocarcinoma, PCPG—Pheochromocytoma and Paraganglioma, PRAD—Prostate Adenocarcinoma, READ—Rectum Adenocarcinoma, SARC—Sarcoma, SKCM—Skin Cutaneious Melanoma, STAD—Stomach Adenocarcinoma, SGCT—Testicular Germ Cell Tumors, THCA—Thyroid Carcinoma, THYM—Thymoma, UCEC—Uterine Corpus Endometrial Carcinoma, UCS—Uterine Carcinosarcoma, UVM—Uveal Melanoma. (1B) A bar chart sorting TCGA mutations to five categories for the study. The x axis depicts the mutation classification according to TCGA*. The y axis depicts the number of mutations in the TCGA mutation categories. The legend depicts the five categories to which the mutations are sorted for this study. *Note: In TCGA, Synonymous mutations are referred to as “Silent”. As the terms are in fact not interchangeable (synonymous mutations are a subcategory of silent mutations) the term “Silent” is replaced with “Synonymous” where needed. (1C) A pie chart of mutation type distribution. The distribution includes all mutations of the 9,915 patients. (1D) Bar chart of polymorphism type distribution. Mutations could be either Single Nucleotide Polymorphisms (SNP), Deletions (DEL) or Insertions (INS). The distribution includes all mutations of the 9,915 patients.

FIG. 2: Flow chart of the study. Center boxes denote preprocessing steps performed for both tasks. Left boxes denote steps performed for the cancer type classification task and right boxes denote steps performed for the survival probability estimation task.

FIGS. 3A-B: A simplified illustration of the feature extraction process. (3A) A representation of the initial genomic information. The X's denote mutations that two patients have in the same gene. The rectangular frames represent the 50-nucleotide-long segments used for the medium resolution features. (3B) An example of the features that would have been extracted for the intron dataset and the UTR dataset according to the initial information shown in 3A.

FIGS. 4A-H: Classification task results. (4A) Bar graph of the F1 scores achieved in the cancer type classification task when using only high-resolution features, high and medium resolution features and all resolutions combined. The scores shown are the average F1 scores achieved by the all-features models across all cancer types. (4B-C) Bar graphs of the F1 scores achieved by the OVA models per cancer type, using features from all levels of resolution with the (4B) unbalanced and (4C) balanced datasets. The x axis depicts the cancer types, the y-axis depicts the F1 scores achieved by the models. Cancer types for which the all-features model outperformed the non-silent model are denoted in grey. Each dataset contained 8,296 features. (4D) Dot blot showing the correlation between the increase in mutational burden and the F1 score improvement obtained by adding silent features to non-silent features. The x-axis depicts the percentage of additional mutational burden that was added on average per patient when adding silent features to non-silent features. The y-axis depicts the percent of improvement gained in F1 score by adding silent features to non-silent features. Every dot represents a single cancer type. (4E) Dot plot of Spearman correlation between Jaccard similarity scores and misclassification rates of pairs of cancer types. Every dot represents a pair of cancer types. The x axis denotes the pair's Jaccard similarity score, and the y axis denotes their misclassification rate. The Spearman coefficient (Rho) and respective p value are noted. (4F) Chart of feature-type distribution of the all-features dataset and of the top ranked features chosen in the classification task. Feature-type distribution of the all-features dataset* (top row), top ranked 100 features (middle row) and top ranked 10 features (bottom row) are depicted. The feature rankings were obtained from the all-features models classifying the 19 cancer types and were averaged across them. The legend (below the image) indicates the enrichment in the amount of each feature-type in the top 10 features compared to its original amount in the all-features dataset (ratio between bottom and top row). *Note: The distribution depicted in the top row is the distribution of the all-features dataset after it underwent preprocessing relevant for the classification task. (4G) Chart of f-type distribution of the balanced all-features dataset and of the top ranked features for the classification task. Feature-type distribution of the all-features dataset (top row), top ranked 100 features (middle row) and top ranked 10 features (bottom row). The feature rankings were obtained from the all-features models and were averaged across cancer types. The legend indicates the enrichment in the amount of each feature-type in the top 10 features when compared to its original amount in the balanced all-features dataset (ratio between bottom and top row). (4H) Polymorphism type distributions in the initial datasets, top 100 features and top 10 features obtained from the OVA models. Each row denotes a model. Within a row, every three clustered columns represent the distribution of the initial dataset (left column), top 100 features (middle column) and top 10 features (right column) of a single cancer type. The analysis was conducted using the feature importance rankings that were obtained from the balanced datasets. The Synonymous models contain only SNPs and thus are excluded from this analysis.

FIG. 5: The number of top 10 ranked genes lists a gene had appeared in when it was mutated by a specific mutation type. The figure is constructed of four panels for readability purposes and is equivalent to a single long panel. Each row in a panel refers to a gene and every column in a panel refers to a mutation type. The results depicted in this figure were obtained from the five single-mutation-type models. Every gene in TCGA that is ranked in the top 10 genes list for at least one cancer type is presented in the figure (the figure includes a total of 216 genes). A lighter shade indicates that the gene was in the top 10 lists of a few cancer types and a darker shade indicates that the gene was in the top 10 lists of many cancer types. The minimum value possible is zero (the gene is not included in the top 10 genes list of any cancer type for that particular model) and the maximum is 19 (the gene is included in the top 10 genes lists for all examined cancers for that particular model).

FIG. 6: The average Spearman correlation of every pair of gene ranking lists of two models. For every cancer type, the correlation between the gene ranking lists of every pair of models was calculated. The average value across cancer types is shown. The respective average p-values are denoted in parentheses. The colors represent the correlation coefficient. A darker color indicates a higher correlation.

FIG. 7: GO terms enrichment for the 19 cancer types. Chart received by using the gene rankings of the all-features models. The figure is constructed of two panels for readability purposes and is equivalent to a single long panel with 113 GO terms. Each row in a panel refers to a GO term and every column in a panel refers to a cancer type. Dark grey positions indicate non-redundant enriched GO terms with a p-value smaller than 0.001 and a q-value (FDR correction) smaller than 0.05. Light grey positions indicate GO terms that are not enriched under these requirements.

FIG. 8: The number of cancer types for which a GO term was enriched using gene rankings from the non-silent models and the all-features models. The figure is constructed of two panels for readability purposes and is equivalent to a single long panel with 123 GO terms. Each row in a panel refers to a GO term and every column in a panel refers to a model from which the gene ranking list was used as input for the GOrilla tool.

FIGS. 9A-B: Survival estimation results. (9A) Line graph of AUC scores achieved by the six RSF models for various times after the initial cancer diagnosis. The x axis depicts the days passed since the diagnosis and the y-axis depicts the AUC score achieved by the models. Each curve denotes a different dataset. The horizontal line depicts the AUC score of a null model. A=All, NS=Non-silent, U=UTR, I=Intron, S=Synonymous, F=Flank, NU=Null. (9B) Feature-type distribution of the all-features dataset and of the top ranked features chosen in the survival probability estimation task. Feature-type distribution of the all-features dataset* (top row), top ranked 100 features (middle row) and top ranked 10 features (bottom row). The feature rankings were obtained from the all-features model. The legend indicates the enrichment in the amount of each feature-type in the top 10 features compared to its original amount in the all-features dataset (ratio between bottom and top row). *Note: The distribution depicted in the top row is the distribution of the all-features dataset after it underwent preprocessing relevant for the survival estimation task.

### DETAILED DESCRIPTION OF THE INVENTION

The present invention, in some embodiments, provides methods of determining a type of cancer in a subject comprising evaluating mutations that are not exonic non-synonymous mutations. Methods of estimating survival time after diagnosis of a subject are also provided. Methods comprising training a machine learning model are also provided.

The invention is based, at least in part, on the first quantitative assessment of the predictive power of silent mutations over cancer classification and prognosis in comparison to non-silent mutations. The results demonstrate the predictive ability of silent mutations to perform both the classification and survival estimation tasks. Moreover, combining both non-silent and silent mutations achieved the best classification results for 68% of the cancer types. When using the same number of features, a combination of silent and non-silent features was still superior to using only non-silent features for 63% of cancer types. For survival estimation the same conclusions are drawn; all silent feature models surpassed the null model for over ten years after an initial diagnosis and combining both silent and non-silent features led to the best survival estimations for more than 9 years. Additionally, silent features were highly ranked in both tasks, surpassing thousands of non-silent features. In fact, considering that numerous silent mutations (which affect gene expression regulation) were found highly predictive by the models and since protein functionality is quite robust to point mutations, it is clear that some of the highly predictive non-silent mutations are such due to their impact on gene expression regulation rather than their impact on protein functionality.

Observing the feature rankings obtained by the different models, it can be seen that low-resolution features are generally ranked higher than high-resolution features, meaning that the number of mutations in an entire functional region of a gene was usually a better predictor than a single specific mutation. This phenomenon is noticed for both silent and non-silent features.

When examining the few silent high-resolution features that were highly ranked, it was not found that they significantly impact mRNA expression levels, splicing or that they had other regulatory effects. However, when examining the low-resolution silent features that were highly ranked, it was found that some contain genomic positions that are assumed to cause a disruption of regulation if mutated. For example, the amount of intronic mutations in the TP53 gene was the second most important feature in the all-features model for detection of LUSC. A SNP mutation was found in the intronic region, 17:7673610: T->C, which annuls a splice site; this mutation was not highly ranked by itself, possibly due to its infrequency (present in only 0.7% of LUSC patients). It is possible that driver mutations could be missed if they are uncommon, even if they have a significant effect. The TP53 gene is maybe the most well-known tumor suppressor and annulling of one of its splice sites could affect tumorigenesis. The number of mutations in the 3′UTR of the SRGAP3 gene was the fourth most important feature in the all-features model for diagnosing SARC. Two deletions, 3: 8985094-8985095: AT and 3: 8985094-8985097: ATAT, were found that both cause the formation of a new miRNA binding site. The first mutation is considerably more common than the second (present in 23.1% and 1.2% of SARC patients respectively) and was in fact the most important mutation in the entire SRGAP3 gene according to the model. The second mutation alone is ranked appreciably lower, unsurprisingly given its low prevalence. The SRGAP3 gene was also reported as a tumor suppressor gene and an addition of a new miRNA binding site could be related to tumorigenesis. The number of intronic mutations in the EGFR gene was ranked the fourth most important gene by the all-features model diagnosing GBM. An insertion in the intronic region, 7: 55020559-55020560: ACACACAC, was found which causes a small but significant decrease in mRNA expression levels (0.7%). This mutation is also uncommon as it is present in only 0.7% of GBM patients. The mutations presented above affect different aspects of the regulation process of known tumor suppressors (TP53, SRGAP3) and oncogenes (EGFR), and could thus influence tumorigenesis. Generally, it seems that there could be many uncommon silent mutations with regulatory affects that are missed for lack of statistical power.

In summary, this study provides abroad, statistical analysis of the predictive abilities of silent and non-silent mutations of various kinds. The results demonstrate that models based on silent mutations are very useful.

By a first aspect, there is provided a method of evaluating a cancer, the method comprising:
a. receiving genomic mutation data from said cancer wherein said data comprises mutations;
b. applying a trained machine learning (ML) model to said received genomic mutation data;
   thereby evaluating a cancer.

By another aspect, there is provided a method of evaluating a cancer, the method comprising receiving genomic mutation data comprising silent mutations and evaluating the silent mutations, thereby evaluating a cancer.

In some embodiments, the method is an in vitro method. In some embodiments, the method is an ex vivo method. In some embodiments, the method is a diagnostic method. In some embodiments, the method is a prognostic method. In some embodiments, the cancer is in a subject. In some embodiments, the cancer is from a subject. In some embodiments, the method is a method of diagnosing the subject. In some embodiments, the method is a method of prognosing the subject.

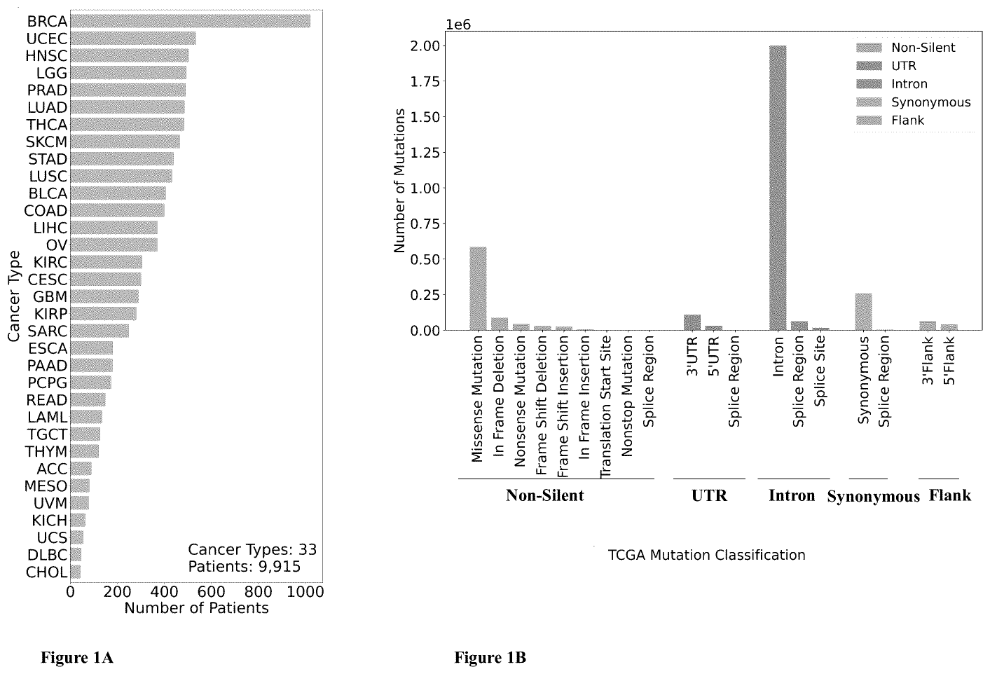

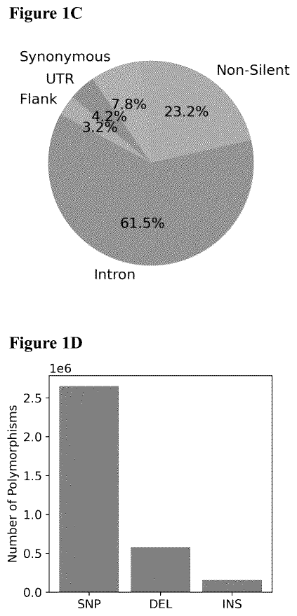

As used herein, the term “cancer” refers to a disease of cell proliferation. In some embodiments, cell proliferation is uncontrolled or overactive cell proliferation. In some embodiments, evaluating a cancer comprises determining the type of cancer. In some embodiments, the type of cancer is the tissue or cell type of origin of the cancer. In some embodiments, the cancer is a solid cancer. In some embodiments, the cancer is a hematopoietic cancer. In some embodiments, the type of cancer is a cancer type provided in FIG. 1. In some embodiments, the cancer type is selected from adrenal cancer, bladder cancer, urothelial cancer, breast cancer, cervical cancer, bile duct cancer, colon cancer, lymphoid cancer, esophageal cancer, brain cancer, head and neck cancer, renal cancer, liver cancer, lung cancer, mesodermal cancer, ovarian cancer, pancreatic cancer, endocrine cancer, neuroendocrine cancer, prostate cancer, rectal cancer, skin cancer, bone cancer, soft tissue cancer, stomach cancer, testicular cancer, thyroid cancer, uterine cancer and uveal cancer. In some embodiments, adrenal cancer is adrenocortical cancer. In some embodiments, adrenal cancer is pheochromocytoma. In some embodiments, cancer is carcinoma. In some embodiments, bladder cancer is bladder urothelial cancer. In some embodiments, breast cancer is breast invasive carcinoma. In some embodiments, the cancer is a squamous cell carcinoma. In some embodiments, the cancer is an adenocarcinoma. In some embodiments, the lymphoma is Lymphoid neoplasm diffuse large B-cell lymphoma. In some embodiments, the brain cancer is a glioma. In some embodiments, the glioma is glioblastoma. In some embodiments, the glioma is a low-grade glioma. In some embodiments, the kidney cancer is kidney chromophobe. In some embodiments, the kidney cancer is kidney renal clear cell carcinoma. In some embodiments, kidney cancer is kidney renal papillary cell carcinoma. In some embodiments, live cancer is liver hepatocellular carcinoma. In some embodiments, lung cancer is mesothelioma. In some embodiments, ovarian cancer is ovarian serous cystadenocarcinoma. In some embodiments, the neuroendocrine cancer is Paraganglioma. In some embodiments, bone cancer is sarcoma. In some embodiments, connective tissue cancer is sarcoma. In some embodiments, skin cancer is melanoma. In some embodiments, melanoma is skin cutaneous melanoma. In some embodiments, testicular cancer is testicular germ cell tumors. In some embodiments, thyroid cancer is thymoma. In some embodiments, uterine cancer is uterine corpus endometrial carcinoma. In some embodiments, the cancer is a carcinosarcoma. In some embodiments, the uveal cancer is uveal melanoma.

In some embodiments, evaluating a cancer comprises estimating survival of the subject after diagnosis. In some embodiments, estimating survival is estimating survival time. In some embodiments, survival is for up to 5, 6, 7, 8, 9 or 10 years from diagnosis. Each possibility represents a separate embodiment of the invention. In some embodiments, survival is for up to 9 years from diagnosis. In some embodiments, survival is for up to 10 years from the diagnosis. In some embodiments, diagnosis is diagnosis of the cancer.

In some embodiments, evaluating a cancer comprises determining the presence of cancer. In some embodiments, the determining the presence of cancer is diagnosis of cancer. In some embodiments, diagnosis is early diagnosis. In some embodiments, presence of cancer is presence of cancer relapse. In some embodiments, determining the presence is determining the return of cancer after therapy. In some embodiments, determining the presence is screening for cancer.

In some embodiments, evaluating a cancer comprises determining a driver mutation in the cancer. In some embodiments, evaluating a cancer comprises determining a disrupted pathway in the cancer. In some embodiments, a pathway is a signaling pathway. In some embodiments, disrupted is as compared to the pathway in a non-cancerous cell. In some embodiments, the non-cancerous cell is of the same cell type or tissue as the cancer.

In some embodiments, evaluating a cancer comprises evaluating a cancer's response to a therapeutic. In some embodiments, evaluating a cancer comprises evaluating a cancer's susceptibility to a therapeutic. In some embodiments, evaluating a cancer comprises testing a therapeutic on the cancer. In some embodiments, the susceptibility is a patient specific susceptibility. In some embodiments, response is patient specific response. In some embodiments, the evaluating is a companion diagnostic.

In some embodiments, the genomic mutation data is DNA sequence data. In some embodiments, the genomic mutation data is data from a biopsy. In some embodiments, the biopsy is a cancer biopsy. In some embodiments, the biopsy is a tumor biopsy. In some embodiments, the biopsy is a liquid biopsy. As used herein, the term “liquid biopsy” refers from a blood sample from a cancer patient where cancer informative information can be isolated. In some embodiments, the cancer informative information is circulating tumor cells. In some embodiments, the informative information is cell free DNA (cfDNA). In some embodiments, the cfDNA is circulating tumor DNA (ctDNA). In some embodiments, the DNA sequence is sequences of cfDNA. In some embodiments, the genomic mutation data is data from cfDNA. In some embodiments, the genomic mutation data is data from cancer cells. In some embodiments, from cancer cells is directly from cancer cells. In some embodiments, cancer cells are cells in the tumor.

In some embodiments, the genomic data comprises mutations. In some embodiments, a mutation is a DNA base or sequence that is different in the cancer as compared to a healthy control. In some embodiments, the healthy control is an atlas of healthy genomic sequences. In some embodiments, the healthy control is a consensus sequence for the species of which the subject is one. In some embodiments, the consensus sequence is a consensus genome. Consensus genomes can be found for example in the NCBI genome browser and the UCSC genome browser. For example, for humans the GRCh38 human genome build can be employed. In some embodiments, the healthy control is a genomic sequence of a healthy individual. In some embodiments, the healthy control is a genomic sequence of a healthy tissue. In some embodiments, the healthy tissue is from the subject that suffers from the cancer. In some embodiments, the healthy tissue is from the subject that provided the genomic mutation data from the cancer. In some embodiments, the mutations are found in the cancer but are absent from healthy tissue of the subject. In some embodiments, the tissue is the same or of the same cell type from which the cancer originated. Thus, it will be understood by a skilled artisan that if for example the cancer is a lung cancer the mutation will not appear in the genome of healthy lung tissue from the subject. Similarly, if the cancer is a breast cancer or skin cancer the mutation would not appear in healthy breast or skin tissue, respectively, from the subject. In some embodiments, a mutation is a point mutation. In some embodiments, a mutation is a deletion. In some embodiments, a mutation is an insertion. In some embodiments, a deletion is a deletion of 1 base. In some embodiments, a deletion is a deletion of 1, 2, 3, 4, or 5 bases. Each possibility represents a separate embodiment of the invention. In some embodiments, an insertion is an insertion of 1 base. In some embodiments, an insertion is an insertion of 1, 2, 3, 4, or 5 bases. Each possibility represents a separate embodiment of the invention.

In some embodiments, the mutation is in a gene. In some embodiments, the mutation is in a gene body. In some embodiments, the mutation is in a gene or proximal to a gene. In some embodiments, proximal is within 10, 9, 8, 7, 6, 5, 4, 3, 2, or 1 kilobase of the gene. Each possibility represents a separate embodiment of the invention. In some embodiments, proximal is within 5000 nucleotides of the gene. In some embodiments, proximal is proximal to the 5′ end of the gene. In some embodiments, the 5′ end of the gene is the 5′ end of the transcribed region. In some embodiments, the 5′ end of the gene is the 5′ end of the 5′ UTR. In some embodiments, proximal is proximal to the 3′ end of the gene. In some embodiments, the 3′ end of the gene is the 3′ end of the transcribed region. In some embodiments, the 3′ end of the gene is the 3′ end of the 3′ UTR. In some embodiments, the proximal is proximal to the 5′ and 3′ end of the gene. In some embodiments, proximal is within 500 nucleotides from the start of the gene and 5000 nucleotides from the end of the gene. In some embodiments, within a distance from a gene is from the transcriptional start site (TSS) and/or the transcriptional termination site (TTS) of the gene. Thus, proximal may be 1 kb upstream and downstream from the start and end of the gene. In some embodiments, the mutation is a silent mutation. As used herein, the term “silent” mutation refers to all mutations that do not directly change a codon that codes for an amino acid into another codon that codes for another amino acid. Most silent mutations will not alter amino acid sequence at all, however it is conceivable that mutations outside of the coding region could actually lead to an alteration in amino acid sequence, such by introducing a premature stop codon, truncation or the like. In some embodiments, the mutation is not an exonic mutation (a mutation in an exon) that is also a non-synonymous mutation. In some embodiments, the genomic mutation data is devoid of non-silent mutations. In some embodiments, the genomic mutation data is devoid of exonic non-synonymous mutations.

The term “codon” refers to a sequence of three DNA or RNA nucleotides that correspond to a specific amino acid or stop signal during protein synthesis. The codon code is degenerate, in that more than one codon can code for the same amino acid. Such codons that code for the same amino acid are known as “synonymous” codons. Thus, for example, CUU, CUC, CUA, CUG, UUA, and UUG are synonymous codons that code for Leucine.

In some embodiments, the mutation is an exonic synonymous mutation. In some embodiments, the mutation is a synonymous mutation. In some embodiments, the mutation is in the coding region but does not alter the amino acid sequence of the encoded protein. In some embodiments, the mutation alters the nucleic acid sequence of a codon to a synonymous nucleic acid sequence. In some embodiments, synonymous mutation data is all mutations found in exons that are synonymous.

In some embodiments, the mutation is an intronic mutation. In some embodiments, the mutation is in an intron. In some embodiments, intron mutation data is all mutations found in introns. In some embodiments, the mutation is in an untranslated region (UTR). In some embodiments, the UTR is the 5′ UTR. As used herein, the term “5′ UTR” refers to the sequence from the transcriptional start site of a gene until the translational start site. Thus, it is all of the 5′ sequence which is transcribed but not translated. In some embodiments, the UTR is the 3′ UTR. As used herein, the term “3′ UTR” refers to the sequence from the translational termination site to the transcriptional termination site. Thus, it is all of the 3′ sequence which is transcribed but not translated. It will be understood that the UTR is gene specific and that some genes have longer and some shorter UTRs. In some embodiments, UTR mutation data is all mutations found in UTR regions. In some embodiments, the UTR is selected from the 3′ and 5′ UTR. In some embodiments, the mutation is in a transcribed region. In some embodiments, the transcribed regions are selected from the UTR, the introns and the exons. In some embodiments, the mutation is in a transcribed region but is not a non-synonymous codon mutation.

In some embodiments, the mutation is in a region flanking a gene. In some embodiments, flanking is proximal to a gene. In some embodiments, flanking is in a non-transcribed region. In some embodiments, flanking is proximal to the gene. In some embodiments, flanking is within 10, 9, 8, 7, 6, 5, 4, 3, 2, or 1 kilobase of the gene. Each possibility represents a separate embodiment of the invention. In some embodiments, flanking is within 5 kilobases of the gene. In some embodiments, flanking comprises 10, 9, 8, 7, 6, 5, 4, 3, 2, or 1 kilobase from the 5′ end of the gene and 10, 9, 8, 7, 6, 5, 4, 3, 2, or 1 kilobase from the 3′ end of the gene. Each possibility represents a separate embodiment of the invention. In some embodiments, flanking comprises the 5000 nucleotides 5′ to the gene and the 5000 nucleotides 3′ to the gene. In some embodiments, flanking comprises the 5000 nucleotides 5′ to the 5′ end of the gene and the 5000 nucleotides 3′ to the 3′ end of the gene. In some embodiments, flanking region mutation data is all mutations found in flanking regions.

In some embodiments, the genomic data is sequencing data. In some embodiments, the sequencing is deep sequencing. In some embodiments, sequencing is next generation sequencing (NGS). In some embodiments, sequencing is whole genome sequencing. In some embodiments, sequencing is whole exome sequencing (WES). In some embodiments, the method further comprises receiving sequencing data from the cancer. In some embodiments, the method further comprises receiving sequencing data from a non-cancerous tissue from the subject. In some embodiments, the non-cancerous tissue is the same tissue from which the cancer originated.

In some embodiments, the genomic mutation data comprises all UTR mutations in the sequencing data. In some embodiments, the genomic mutation data comprises all intronic mutations from the sequencing data. In some embodiments, the genomic mutation data comprises all flanking region mutations in the sequencing data. In some embodiments, the genomic mutation data comprises all synonymous exonic mutations in the sequencing data. In some embodiments, the sequencing data is from the subject. In some embodiments, the sequencing data is from cancer patients.

In some embodiments, the genomic mutation data further comprises non-synonymous mutations. In some embodiments, the genomic mutation data further comprises exonic non-synonymous mutations. In some embodiments, non-synonymous mutation data is all non-synonymous mutations found in exons. In some embodiments, the genomic mutation further comprises non-silent mutations. In some embodiments, the genomic mutation data comprises all mutations. In some embodiments, all mutations is all mutations in the sequencing data. In some embodiments, the sequencing data is from the subject. In some embodiments, the sequencing data is from cancer patients. In some embodiments, the sequencing data is all mutations found in WES data from the subject. In some embodiments, the sequencing data is all mutations found in WES data from the cancer patients.

In some embodiments, the genomic mutation data comprises intronic mutations and the cancer is selected from cervical cancer, colon cancer, brain cancer, renal cancer, and liver cancer. In some embodiments, the genomic mutation data consists of intronic mutations, and the cancer is selected from cervical cancer, colon cancer, brain cancer, renal cancer, and liver cancer.

In some embodiments, the genomic mutation data comprises UTR mutations and the cancer is selected from cervical cancer, bone cancer and soft tissue cancer. In some embodiments, the genomic mutation data consists of UTR mutations and the cancer is selected from cervical cancer, bone cancer and soft tissue cancer.

In some embodiments, the genomic mutation data comprises flanking region mutations and the cancer is selected from cervical cancer, bone cancer and soft tissue cancer. In some embodiments, the genomic mutation data consists of flanking region mutations and the cancer is selected from cervical cancer, bone cancer and soft tissue cancer.

In some embodiments, the genomic mutation data comprises UTR mutations, intronic mutations, flanking region mutations and synonymous mutations and the cancer is selected from bladder cancer, urothelial cancer, breast cancer, cervical cancer, colon cancer, renal cancer, liver cancer, lung cancer, ovarian cancer, bone cancer, soft tissue cancer, skin cancer, thyroid cancer and uterine cancer. In some embodiments, the genomic mutation data consists of UTR mutations, intronic mutations, flanking region mutations and synonymous mutations and the cancer is selected from bladder cancer, urothelial cancer, breast cancer, cervical cancer, colon cancer, renal cancer, liver cancer, lung cancer, ovarian cancer, bone cancer, soft tissue cancer, skin cancer, thyroid cancer and uterine cancer.

In some embodiments, the genomic mutation data comprises UTR mutations, intronic mutations, flanking region mutations and synonymous mutations and the cancer is selected from breast cancer, colon cancer, brain cancer, renal cancer, liver cancer, ovarian cancer, bone cancer, soft tissue cancer, thyroid cancer and uterine cancer. In some embodiments, the genomic mutation data consists of UTR mutations, intronic mutations, flanking region mutations and synonymous mutations and the cancer is selected from breast cancer, colon cancer, brain cancer, renal cancer, liver cancer, ovarian cancer, bone cancer, soft tissue cancer, thyroid cancer and uterine cancer.

In some embodiments, the genomic mutation data comprises UTR mutations, intronic mutations, flanking region mutations, non-synonymous mutations and synonymous mutations and the cancer is selected from bladder cancer, urothelial cancer, breast cancer, cervical cancer, colon cancer, renal cancer, liver cancer, lung cancer, ovarian cancer, bone cancer, soft tissue cancer, skin cancer, thyroid cancer and uterine cancer. In some embodiments, the genomic mutation data consists of UTR mutations, intronic mutations, flanking region mutations, non-synonymous mutations and synonymous mutations and the cancer is selected from bladder cancer, urothelial cancer, breast cancer, cervical cancer, colon cancer, renal cancer, liver cancer, lung cancer, ovarian cancer, bone cancer, soft tissue cancer, skin cancer, thyroid cancer and uterine cancer.

In some embodiments, the genomic mutation data comprises UTR mutations, intronic mutations, flanking region mutations, synonymous mutations and non-synonymous mutations and the cancer is selected from breast cancer, colon cancer, brain cancer, renal cancer, liver cancer, ovarian cancer, bone cancer, soft tissue cancer, thyroid cancer and uterine cancer. In some embodiments, the genomic mutation data consists of UTR mutations, intronic mutations, flanking region mutations, synonymous mutations and non-synonymous mutations and the cancer is selected from breast cancer, colon cancer, brain cancer, renal cancer, liver cancer, ovarian cancer, bone cancer, soft tissue cancer, thyroid cancer and uterine cancer.

In some embodiments, the evaluating comprises employing a machine learning (ML) algorithm. In some embodiments, the evaluating comprises applying a ML algorithm to the genomic mutation data. In some embodiments, predicted is predicted by the machine learning algorithm. In some embodiments, the ML algorithm is a trained algorithm. In some embodiments, the machine learning algorithm is the machine learning algorithm during training. In some embodiments, the algorithm is trained to evaluate cancer. In some embodiments, the algorithm is trained to determine the type of cancer. In some embodiments, the type of cancer is the tissue or cell type of origin of the cancer. In some embodiments, the algorithm is trained to estimate the survival time of the subject suffering from the cancer.

In some embodiments, the algorithm is a classifier. In some embodiments, the algorithm classifies the cancer. In some embodiments, the classifier classifies the subject. In some embodiments, the classification is to a type of cancer. In some embodiments, the classification is to an estimated time of survival.

In some embodiments, the machine learning algorithm is a machine learning model. In some embodiments, the machine learning model implements a machine learning algorithm. In some embodiments, the algorithm is a classifier. In some embodiments, the algorithm is a regression model. In some embodiments, the algorithm is supervised. In some embodiments, the algorithm is unsupervised. In some embodiments, the machine learning algorithm is trained on a training set. In some embodiments, a trained machine learning algorithm is applied to genomic mutation data from the subject.

In some embodiments, the training set comprising the genomic mutation data from cancer patients. In some embodiments, the cancer patients have a known cancer type. In some embodiments, the method is a method of determining cancer type and the training set comprises cancer patients with a known cancer type. In some embodiments, the training set further comprises labels. In some embodiments, the labels identify the cancer type of the cancer. In some embodiments, the labels identify the cancer type of the cancers from the cancer patients. In some embodiments, the ML model outputs a classification of the cancer in the subject. In some embodiments, the classification is as one of the known cancer types. In some embodiments, the machine learning algorithm is trained on the genomic mutation data from cancer patients with a cancer of a first type and genomic mutation data from cancer patients with a cancer of a second type.

In some embodiments, the cancer patients have a known survival time. In some embodiments, survival time is survival time after diagnosis. In some embodiments, survival time is estimated survival time. In some embodiments, the method is a method of determining survival time and the training set comprises cancer patients with a known survival time. In some embodiments, the training set further comprises labels. In some embodiments, the labels identify the survival time of the subject with cancer. In some embodiments, the ML model outputs an estimated survival time of the subject. In some embodiments, the machine learning algorithm is trained on the genomic mutation data from cancer patients with a first survival time and genomic mutation data from cancer patients with a second survival time.

In some embodiments, the training set comprises healthy subject. In some embodiments, healthy subjects are subjects that do not have cancer. In some embodiments, the cancer patients have a known survival time. In some embodiments, the method is a method of diagnosing cancer and the training set comprises cancer patients and healthy subjects. In some embodiments, the training set further comprises labels. In some embodiments, the labels identify the subject as healthy or suffering from cancer. In some embodiments, the ML model outputs a diagnosis of cancer or healthy. In some embodiments, the ML model outputs a diagnostic cancer score. In some embodiments, the score is proportional to the likelihood of the subject suffering from cancer. In some embodiments, the machine learning algorithm is trained on the genomic mutation data from cancer patients and healthy patients.

In some embodiments, a healthy subject is a subject recovered from cancer. In some embodiments, a healthy subject is a subject that previously suffered from cancer. In some embodiments, the training set comprises genomic mutation data from a subject a time when the subject has cancer and from the same subject at a time when the subject does not have cancer. In some embodiments, the genomic mutation data is from the organ which previously was cancerous.

In some embodiments, the cancer patients have a cancer with a known driver mutation. In some embodiments, in some embodiments, driver mutation is a plurality of driver mutations. In some embodiments, the method is a method of determining a driver mutation and the training set comprises cancer patients with a cancer with a known driver mutation. In some embodiments, the training set further comprises labels. In some embodiments, the labels identify the driver mutation of the cancer. In some embodiments, the ML model outputs a predicted driver mutation. In some embodiments, the machine learning algorithm is trained on the genomic mutation data from cancer patients with a first driver mutation and genomic mutation data from cancer patients with a second driver mutation.

In some embodiments, the cancer patients have a known responsiveness to a cancer therapy. In some embodiments, the cancer patients comprise patients that respond to the therapy (responders) and subjects that do not respond to the therapy (non-responders). In some embodiments, the method is a method of determining responsiveness to a therapy and the training set comprises responders and non-responders. In some embodiments, the training set further comprises labels. In some embodiments, the labels identify the responsiveness of the patient. In some embodiments, the labels identify the patient as responsive or non-responsive to the therapy. In some embodiments, the ML model outputs “responsive” or “non-responsive”. In some embodiments, the ML model outputs a responsiveness score. In some embodiments, the score is proportional to the likelihood of the subject being a responder. In some embodiments, the score is proportional to the likelihood of the subject being a non-responder. In some embodiments, the machine learning algorithm is trained on the genomic mutation data from responders and non-responders.

In some embodiments, the therapy is a cancer therapy. In some embodiments, the therapy is an anticancer therapy. In some embodiments, the therapy comprises administering to the patient a therapeutic agent. Therapies for treating cancer are well known in the art and responsiveness to any such therapy may be tested by a method of the invention. Examples of cancer therapies include, but are not limited to: radiation therapy, chemotherapy, targeted therapy, immunotherapy, adoptive immune cell transfer therapy and surgery. These therapies and the agents used in these therapies are well known to a skilled artisan and response to any of them may be tested by a method of the invention.

In some embodiments, the training set comprises mutations. In some embodiments, mutations are genomic mutation data. In some embodiments, the mutations are from cancer patients. In some embodiments, the training set comprises genomic mutation data from cancer patients. In some embodiments, cancer patients is a plurality of cancer patients. In some embodiments, the training set further comprises mutations from healthy patients. In some embodiments, the mutations are mutations that appear in at least two of the cancer patients. In some embodiments, the mutations are mutations that appear in a plurality of the cancer patients. In some embodiments, the mutations consist of mutations that appear in at least two of the cancer patients. In some embodiments, the training set is produced by providing mutations from cancer patients and selected for the training set mutations that appear in at least two of the cancer patients. In some embodiments, in at least two of the cancer patients is in the genomic mutation data from at least two of the cancer patients.

In some embodiments, the training set comprises received genomic mutation data. In some embodiments, the training set comprises received genomic mutation data in both cancer patients with a first cancer type and cancer patients with a second cancer type. In some embodiments, the training set comprises received genomic mutation data in both cancer patients with a first survival time and cancer patients with a second survival time. In some embodiments, the training set comprises received genomic mutation data in both cancer patients and healthy patients. In some embodiments, the training set comprises received genomic mutation data in both cancer patients with a cancer with a first driver mutation and cancer patients with a cancer with a second driver mutation. In some embodiments, the training set comprises received genomic mutation data in both cancer patients that are responsive and cancer patients that are non-responsive to a therapy. It will be understood by a skilled artisan that while a first and second are recited the training set may also include a third, fourth, fifth, etc. In some embodiments, the training set comprises cancer patients with at least two different types of cancer. In some embodiments, the training set comprises cancer patients with cancer with at least two different driver mutations. In some embodiments, at least two different types of cancer is at least 2, 3, 4, 5, 6, 7, 8, 9, 10, 11, 12, 13, 14, 15, 16, 17, 18, or 19 types of cancer. Each possibility represents a separate embodiment of the invention. In some embodiments, at least two different driver mutations is at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900 or 100 driver mutations. Each possibility represents a separate embodiment of the invention. In some embodiments, at least two different types of cancer is at least 5 types of cancer. In some embodiments, at least two different types of cancer is at least 10 types of cancer. In some embodiments, at least two different types of cancer is at least 15 types of cancer. In some embodiments, at least two different types of cancer is at least 19 types of cancer. In some embodiments, the training set comprises received genomic mutation data for only one silent mutation type. In some embodiments, the mutation types are selected from UTR mutations, flanking region mutations, intronic mutations and synonymous exonic mutations. In some embodiments, the training set comprises received genomic mutation data for all types of silent mutations. In some embodiments, the training set comprises received genomic mutation data for all types of silent mutations and non-silent mutations. In some embodiments, non-silent mutations are exonic, non-synonymous mutations. In some embodiments, the training set comprises labels. In some embodiments, the labels are associated with the cancer type of the patients. In some embodiments, the labels are associated with the survival times of the patients. In some embodiments, the mutations are labeled with the labels. In some embodiments, the genomic mutation data is labeled with the labels.

In some embodiments, at an inference stage the trained machine learning algorithm is applied. In some embodiments, the trained machine learning algorithm is applied to mutation from the subject. In some embodiments, the trained machine learning algorithm is applied to genomic mutation data from the subject.

In some embodiments, at the inference stage an input is received. In some embodiments, the input comprises the mutations in the subject. In some embodiments, the input comprises the genomic mutation data from the subject. In some embodiments, in the subject is in the cancer of the subject. In some embodiments, from the subject is from the cancer of the subject. In some embodiments, in the subject or from the subject is in or from a sample from the subject. In some embodiments, the sample comprises cancer cells. In some embodiments, the sample comprises DNA. In some embodiments, the DNA is cancer DNA. In some embodiments, the sample is a tumor sample. In some embodiments, the sample is a biopsy. In some embodiments, the sample is a liquid biopsy. In some embodiments, the sample is a bodily fluid. In some embodiments, a bodily fluid is selected from blood, serum, plasma, gastric fluid, intestinal fluid, saliva, bile, tumor fluid, breast milk, urine, interstitial fluid, cerebral spinal fluid and stool. In some embodiments, the bodily fluid is blood or plasma. In some embodiments, the fluid is a fluid that contains cancer cells. In some embodiments, the fluid is a fluid that contains cell free DNA (cfDNA). In some embodiments, the cfDNA comprises cancer cfDNA. In some embodiments, the bodily fluid is selected from: blood, serum, plasma, gastric fluid, intestinal fluid, saliva, bile, tumor fluid, breast milk, urine, interstitial fluid, cerebral spinal fluid and stool. In some embodiments, the fluid is blood or plasma. In some embodiments, the subject suffers from cancer. In some embodiments, the cancer type of the subject is unknown. some embodiments, at the inference stage the trained machine learning algorithm is applied. In some embodiments, applied is applied to the input. In some embodiments, the input is the received input. In some embodiments, the inference stage is to predict cancer type. In some embodiments, the inference stage is to estimate survival time. In some embodiments, estimate is predict.

In some embodiments, the machine learning algorithm outputs a cancer type. In some embodiments, the cancer type is one of the cancer types of the cancer patients in the training set. In some embodiments, the cancer type is selected from one of the cancers provided hereinabove. In some embodiments, the machine learning algorithm outputs a survival time. In some embodiments, a survival time is a survival window. In some embodiments, a window is 1, 2, 3, 4, 5, 6, 7, 8, 9, 10, 11, 12, 18, or 24, months. Each possibility represents a separate embodiment of the invention. In some embodiments, the survival time is selected from 1, 2, 3, 4, 5, 6, 7, 8, 9, 10, 11, 12, 18, 24, 30, 36, 42, 48, 54, 60, 66, 72, 78, 84, 90, 96, 102, 108, 114, and 120 months. Each possibility represents a separate embodiment of the invention. In some embodiments, the survival time is selected from at least 1, 2, 3, 4, 5, 6, 7, 8, 9, 10, 11, 12, 18, 24, 30, 36, 42, 48, 54, 60, 66, 72, 78, 84, 90, 96, 102, 108, 114, and 120 months. Each possibility represents a separate embodiment of the invention. In some embodiments, the survival time is selected from at most 1, 2, 3, 4, 5, 6, 7, 8, 9, 10, 11, 12, 18, 24, 30, 36, 42, 48, 54, 60, 66, 72, 78, 84, 90, 96, 102, 108, 114, 120 months or beyond 120 months. Each possibility represents a separate embodiment of the invention.

In some embodiments, the machine learning model is a machine learning algorithm. In some embodiments, the algorithm is a supervised learning algorithm. In some embodiments, the algorithm is an unsupervised learning algorithm. In some embodiments, the algorithm is a reinforcement learning algorithm. In some embodiments, the machine learning model is a Convolutional Neural Network (CNN). In some embodiments, the machine learning model is a decision tree model. In some embodiments, the classifier is a decision tree classifier or classification tree. In some embodiments, the machine learning is decision tree learning. In some embodiments, the decision tree is a regression tree. Decision tree algorithms are well known in the art and include, for example, LightGBM, random survival forest model, ID3, C4.5, CART, CHAID and MARS, although any such algorithm may be employed. In some embodiments, the ML model comprises LightGBM. In some embodiments, the ML model comprises random survival forest model. In some embodiments, the at least one hardware processor trains a machine learning model. In some embodiments, the model is based, at least in part, on a training set. In some embodiments, the model is based on a training set. In some embodiments, the model is trained on a training set. In some embodiments, the at least one hardware processor applies the machine learning model to genomic mutation data from a subject.

By another aspect, there is provided a method of evaluating a cancer, the method comprising receiving a sample from said cancer and detecting a mutation in a gene selected from those discovered herein, thereby evaluating cancer.

In some embodiments, the method is a method of determining a driver mutation in the cancer. In some embodiments, the method is a method of determining a driver gene in the cancer. In some embodiments, the gene is the gene comprising the driver mutation. In some embodiments, evaluating comprises determining a driver gene, driver mutation or both. In some embodiments, evaluating is predicting survival. In some embodiments, evaluating is prognosing the cancer. In some embodiments, prognosing is prognosing the subject. In some embodiments, the subject is the subject with the cancer. In some embodiments, the subject is the subject that provided the sample. In some embodiments, the sample is a sample comprising DNA. In some embodiments, the detecting is detecting in the DNA.

In some embodiments, the method is a method of predicting subject survival. In some embodiments, the method is a method of estimating subject survival. In some embodiments, prognosis comprises survival. In some embodiments, evaluating comprises predicting or estimating survival. In some embodiments, the mutations for use in evaluating survival are provided hereinbelow. In some embodiments, the mutation for use in evaluating survival are provided in Gutman et al. In some embodiments, Gutman et al. is Supplementary Data 3 of Gutman et al., hereby incorporated by reference in its entirety.

In some embodiments, the mutation is a silent mutation. In some embodiments, the mutation is a mutation provided hereinbelow. In some embodiments, the mutation is a mutation provided in Gutman et al., 2021, “Estimating the predictive power of silent mutations on cancer classification and prognosis”, NPJ Genome Med., August 12; 6(1):67, herein incorporated by reference in its entirety. In some embodiments, in Gutman et al., is in Supplementary Data 2 of Gutman et al., herein incorporated by reference in its entirety. In some embodiments, the mutation is in a gene provided hereinbelow. In some embodiments, the mutation is in a gene provided in Gutman et al. In some embodiments, the silent mutation is of the type provided in Table 5. In some embodiments, the silent mutation is of the type provided in Gutman et al.

In some embodiments, the cancer is a cancer provided in Table 5. In some embodiments, the cancer is a cancer type provided in Table 5. In some embodiments, the cancer is selected from a cancer or cancer type provided in Table 5 and the gene is selected from the genes provided for that cancer or cancer type in Table 5. In some embodiments, the cancer is selected from a cancer or cancer type provided in Table 5 and the mutation type is selected from the mutation type provided for that cancer or cancer type in Table 5. In some embodiments, the gene and mutation type are selected from those in Table 5 provided for that cancer or cancer type. In some embodiments, the cancer is a cancer provided in Gutman et al. In some embodiments, the cancer and corresponding mutation or gene comprising the mutation is provided in Gutman et al. In some embodiments, the cancer and corresponding mutation type is provided in Gutman et al. In some embodiments, the cancer and corresponding mutation type for a specific mutation or gene comprising the mutation is provided in Gutman et al.

In some embodiments, the cancer is a bladder cancer and the mutation is in an intron of KMT2C, SET, SEPT9, PRKAR1A, DCC, CNTNAP2, ELF3, PDE4D, TET2, or PARG. Each possibility represents a separate embodiment of the invention. In some embodiments, the cancer is a bladder cancer and the mutation is in a UTR of PABPC1, FAM46C, PAX3, BCR, TRIM2, TEC, HLA-A, PAX3, LARP4, or HMGN2. Each possibility represents a separate embodiment of the invention. In some embodiments, the cancer is a bladder cancer and the mutation is in a flanking region of DUX4, MLLT4, U2, AK2, RGPD3, LARP4, BCR, WT1, MALAT1, or CRTC1. Each possibility represents a separate embodiment of the invention. In some embodiments, the cancer is a bladder cancer and the mutation is a exonic synonymous mutation in MUC4, AK2, MUC16, CHEK2, KMT2C, LHFP, HLA-A, AK2, CHEK2, or WHSC1L1. Each possibility represents a separate embodiment of the invention. In some embodiments, the bladder cancer is BLCA.

In some embodiments, the cancer is a breast cancer and the mutation is in an intron of PAX3, TFG, CR1, BCLAF1, CTNNA2, KMT2C, SLC34A2, ZNF521, DDR2, or CR1. Each possibility represents a separate embodiment of the invention. In some embodiments, the cancer is a breast cancer and the mutation is in a UTR of TEC, HIP1, CAMTA1, PAX3, EIF3E, LARP4, HLA-A, TBL1X, PHOX2B, or PPP6C. Each possibility represents a separate embodiment of the invention. In some embodiments, the cancer is a breast cancer and the mutation is in a flanking region of DUX4, U2, YOD1, MALAT1, ZNRF3, MLLT4, AK2, RGPD3, MUC1, or SGK1. Each possibility represents a separate embodiment of the invention. In some embodiments, the cancer is a breast cancer and the mutation is a exonic synonymous mutation in MUC4, AK2, HOXA1, MUC16, CLIP1, USP8, KMT2C, CREBBP, TSHR, or CHEK2. Each possibility represents a separate embodiment of the invention. In some embodiments, the breast cancer is BRCA.

In some embodiments, the cancer is a cervical cancer and the mutation is in an intron of CARD11, PAX3, SEPT9, RGPD3, ZNRF3, ACSL3, DDR2, PIK3CB, MLLT4, or PDGFB. Each possibility represents a separate embodiment of the invention. In some embodiments, the cancer is a cervical cancer and the mutation is in a UTR of PABPC1, BCR, CSF1, FOXO3, RNF4, SRGAP3, LARP4, TRIM2, RARA, or PDGFB. Each possibility represents a separate embodiment of the invention. In some embodiments, the cancer is a cervical cancer and the mutation is in a flanking region of RGPD3, BCR, PRDM16, U2, DUX4, NR4A3, WWTR1, USP6, AK2, or DDX6. Each possibility represents a separate embodiment of the invention. In some embodiments, the cancer is a cervical cancer and the mutation is a exonic synonymous mutation in MUC4, KMT2C, HLA-A, CHEK2, MUC16, AK2, RANBP2, TF, NUP98, or BRD3. Each possibility represents a separate embodiment of the invention. In some embodiments, the cervical cancer is CESC.

In some embodiments, the cancer is a colon cancer and the mutation is in an intron of CNTRL, PARG, TPR, KMT2C, STAG2, PTPRT, FUS, ROBO2, KIAA1598, or PAX3. Each possibility represents a separate embodiment of the invention. In some embodiments, the cancer is a colon cancer and the mutation is in a UTR of FAM46C, EIF3E, TEC, BCL11A, MLLT11, BCR, FST, UBR5, MUC16, or TRIM2. Each possibility represents a separate embodiment of the invention. In some embodiments, the cancer is a colon cancer and the mutation is in a flanking region of YOD1, U2, DUX4, ZNRF3, AK2, MALAT1, MYH1, NFATC2, ALK, or LCP1. Each possibility represents a separate embodiment of the invention. In some embodiments, the cancer is a colon cancer and the mutation is a exonic synonymous mutation in RANBP2, AK2, MUC4, CLIP1, MAP3K13, RANBP2, KMT2C, FANCD2, MUC16, or CHEK2. Each possibility represents a separate embodiment of the invention. In some embodiments, the colon cancer is COAD.

In some embodiments, the cancer is a brain cancer and the mutation is in an intron of PTPRT, RGS7, CSMD3, PARG, CR1, LRP1B, KMT2C, SET, SEPT9, PBRM1, EZH2, PARG, CR1, EGFR, ARHGAP26, CSMD3, SEPT9, DDR2, COL1A1, or CBLC. Each possibility represents a separate embodiment of the invention. In some embodiments, the cancer is a brain cancer and the mutation is in a UTR of PAX3, PABPC1, FAM46C, HIP1, TRIM2, EBF1, BCR, RSPO3, SRGAP3, RNF4, LARP4, PABPC1, FAM46C, BCL11A, CCR7, QKI, CSMD3, SDHA, FGFR3, or MSN. Each possibility represents a separate embodiment of the invention. In some embodiments, the cancer is a brain cancer and the mutation is in a flanking region of DUX4, U2, AK2, MLLT4, MALAT1, RGPD3, BCR, GATA3, RDM1, RGS7, U2, MLLT4, LARP4, MDS2, AK2, DUX4, RGPD3, MALAT1, FANCD2, or WWP2. Each possibility represents a separate embodiment of the invention. In some embodiments, the cancer is a brain cancer and the mutation is a exonic synonymous mutation in MUC4, FAM135B, CSMD3, DCC, HOXA1, NSD1, MUC16, CHEK2, FAT4, CTNND2, MUC16, TF, MECOM, KMT2C, MAP3K13, EGFR, AFF4, AK2, or FAT1. Each possibility represents a separate embodiment of the invention. In some embodiments, the brain cancer is GBM. In some embodiments, the brain cancer is LGG.

In some embodiments, the cancer is a head and neck cancer and the mutation is in an intron of KMT2C, PARG, SET, PAX3, PAX5, SEPT9, CNTNAP2, TET2, CARD11, or PTPRT. Each possibility represents a separate embodiment of the invention. In some embodiments, the cancer is a head and neck cancer and the mutation is in a UTR of PABPC1, PAX3, TBL1X, FAM46C, BCR, IL7R, CBL, CAMTA1, EIF3E, or SDHA. Each possibility represents a separate embodiment of the invention. In some embodiments, the cancer is a head and neck cancer and the mutation is in a flanking region of DUX4, MLLT4, AK2, RGPD3, U2, CRTC1, MALAT1, DDX6, MECOM, or CRTC1. Each possibility represents a separate embodiment of the invention. In some embodiments, the cancer is a head and neck cancer and the mutation is a exonic synonymous mutation in MUC4, MUC16, CLIP1, KMT2C, CHEK2, HLA-A, PABPC1, BRD3, MECOM, or FANCD2. Each possibility represents a separate embodiment of the invention. In some embodiments, the head and neck cancer is HNSC.

In some embodiments, the cancer is a renal cancer and the mutation is in an intron of SET, PCM1, MUC16, PDE4D, RGPD3, KMT2C, CSMD3, SEPT9, ALB, CNTNAP2, EZH2, FAS, SETBP1, SMARCA4, PTPN6, MLLT4, CSMD3, F5, KMT2C, or MUC16. Each possibility represents a separate embodiment of the invention. In some embodiments, the cancer is a renal cancer and the mutation is in a UTR of GNAS, PHOX2B, CAMTA1, FAM46C, PHOX2B, SRGAP3, CAMTA1, PLAG1, ARHGEF12, EIF3E, IL7R, LARP4, EIF3E, SDHA, BCL11A, IL2, TRIM2, BCR, DCT, or ERCC2. Each possibility represents a separate embodiment of the invention. In some embodiments, the cancer is a renal cancer and the mutation is in a flanking region of MUC1, U2, DUX4, AK2, RGPD3, H3F3B, MALAT1, BCR, DDX6, MLLT4, AK2, U2, LARP4, DUX4, MALAT1, RGPD3, ZNRF3, YOD1, STK11, or MLLT4. Each possibility represents a separate embodiment of the invention. In some embodiments, the cancer is a renal cancer and the mutation is a exonic synonymous mutation in MUC16, MUC4, KMT2C, CHEK2, NUTM2A, PER1, NUP98, HLA-A, SDHA, PER1, KMT2C, FAT3, FAT1, ROBO2, PICALM, RANBP2, MUC4, BRCA2, PARG, or ARID2. Each possibility represents a separate embodiment of the invention. In some embodiments, the renal cancer is KIRC. In some embodiments, the renal cancer is KIRP.

In some embodiments, the cancer is a liver cancer and the mutation is in an intron of SET, SDHA, SEPT9, ALB, CDC73, DDX3X, MUC16, PARG, HLA-A, or PTPRC. Each possibility represents a separate embodiment of the invention. In some embodiments, the cancer is a liver cancer and the mutation is in a UTR of GNAS, FAM46C, PHOX2B, PRRX1, EIF3E, CAMTA1, SRGAP3, SDHA, MLLT11, or PABPC1. Each possibility represents a separate embodiment of the invention. In some embodiments, the cancer is a liver cancer and the mutation is in a flanking region of DUX4, MUC1, YOD1, TERT, MALAT1, MLLT4, CRTC1, AK2, U2, or ZNRF3. Each possibility represents a separate embodiment of the invention. In some embodiments, the cancer is a liver cancer and the mutation is a exonic synonymous mutation in MUC4, MUC16, RANBP2, FCGR2B, SETBP1, TAL1, KMT2C, MAP3K13, AK2, or CHEK2. Each possibility represents a separate embodiment of the invention. In some embodiments, the liver cancer is LIHC.

In some embodiments, the cancer is a lung cancer and the mutation is in an intron of PARG, SET, CDH10, CSMD3, KMT2C, PAX3, FANCD2, CTNND2, SEPT9, FAM135B, KMT2C, SET, LRP1B, SEPT9, CSMD3, FHIT, PDE4D, PARG, CDH10, or CNTNAP2. Each possibility represents a separate embodiment of the invention. In some embodiments, the cancer is a lung cancer and the mutation is in a UTR of FAM46C, PABPC1, CAMTA1, EIF3E, SDHA, FGFR3, CDH10, HLA-A, PHOX2B, FST, PABPC1, PAX3, BCR, SDHA, RSPO3, EIF3E, PIK3R1, HMGN2, or HLA-A. Each possibility represents a separate embodiment of the invention. In some embodiments, the cancer is a lung cancer and the mutation is in a flanking region of MLLT4, U2, RGPD3, AK2, MALAT1, ZNRF3, CRTC1, LARP4B, CDKN1A, DUX4, MLLT4, U2, AK2, RGPD3, MALAT1, SP1, SGK1, CDKN1A, or BCR. Each possibility represents a separate embodiment of the invention. In some embodiments, the cancer is a lung cancer and the mutation is a exonic synonymous mutation in CDH10, MUC16, KMT2C, PABPC1, CSMD3, CHEK2, AK2, MECOM, FAT1, MUC4, MUC16, NUP98, AK2, KMT2C, FAT3, RNF43, HLA-A, CHEK2, or CSMD3. Each possibility represents a separate embodiment of the invention. In some embodiments, the lung cancer is LUAD. In some embodiments, the lung cancer is LUSC.

In some embodiments, the cancer is an ovarian cancer and the mutation is in an intron of ARHGAP26, DDX6, ANK1, NCOA1, MYH11, SF3B1, CASP8, HLA-A, MUC16, or NACA. Each possibility represents a separate embodiment of the invention. In some embodiments, the cancer is an ovarian cancer and the mutation is in a UTR of PAX3, FAM46C, SDHA, TEC, PABPC1, CAMTA1, QKI, MLLT11, MAF, or JAK3. Each possibility represents a separate embodiment of the invention. In some embodiments, the cancer is an ovarian cancer and the mutation is in a flanking region of AK2, MALAT1, U2, MLLT4, DUX4, MDS2, MUC16, PRDM16, RGPD3, or STK11. Each possibility represents a separate embodiment of the invention. In some embodiments, the cancer is an ovarian cancer and the mutation is a exonic synonymous mutation in MUC4, HOXA1, AMER1, MUC16, CLIP1, RANBP2, KMT2C, SDHA, CHD4, or STIL. Each possibility represents a separate embodiment of the invention. In some embodiments, the ovarian cancer is OV.

In some embodiments, the cancer is a prostate cancer and the mutation is in an intron of KMT2C, CNTNAP2, TG, CR1, SET, NTRK3, PARG, PTPRT, RGPD3, or LRP1B. Each possibility represents a separate embodiment of the invention. In some embodiments, the cancer is a prostate cancer and the mutation is in a UTR of FAM46C, PAX3, PABPC1, PPP6C, TEC, SDHA, BCR, HIP1, BCL2, or EPHA3. Each possibility represents a separate embodiment of the invention. In some embodiments, the cancer is a prostate cancer and the mutation is in a flanking region of DUX4, LARP4, MLLT4, U2, MALAT1, AK2, RGPD3, WWP2, SDHC, or PRDM16. Each possibility represents a separate embodiment of the invention. In some embodiments, the cancer is a prostate cancer and the mutation is a exonic synonymous mutation in MUC4, KMT2C, CHEK2, UBR5, MUC16, APOB, TRRAP, PTPRT, CNTNAP2, or CSMD3. Each possibility represents a separate embodiment of the invention. In some embodiments, the prostate cancer is PRAD.

In some embodiments, the cancer is a bone or soft tissue cancer and the mutation is in an intron of CARD11, PAX3, HLA-A, RGPD3, ZNRF3, CR1, CTCF, SEPT9, PARG, or CTNNA2. Each possibility represents a separate embodiment of the invention. In some embodiments, the cancer is a bone or soft tissue cancer and the mutation is in a UTR of BCR, SRGAP3, PABPC1, CSF1, RNF4, FOXO3, NF1, TRIM2, PLAG1, or ZNRF3. Each possibility represents a separate embodiment of the invention. In some embodiments, the cancer is a bone or soft tissue cancer and the mutation is in a flanking region of NR4A3, RGPD3, BCR, PRDM16, SP1, DUX4, USP6, PAX8, MLLT4, or DDX6. Each possibility represents a separate embodiment of the invention. In some embodiments, the cancer is a bone or soft tissue cancer and the mutation is a exonic synonymous mutation in MUC4, RANBP2, KMT2C, CHEK2, AK2, NUP98, HLA-A, MECOM, PABPC1, or MUC16. Each possibility represents a separate embodiment of the invention. In some embodiments, the bone or soft tissue cancer is SARC. In some embodiments, a bone or soft tissue cancer is a sarcoma.

In some embodiments, the cancer is a skin cancer and the mutation is in an intron of PTPRT, MYH1, PARG, C6, KMT2C, C3, MUC16, SET, LRP1, or SLC34A2. Each possibility represents a separate embodiment of the invention. In some embodiments, the cancer is a skin cancer and the mutation is in a UTR of PABPC1, CAMTA1, TRIM2, CD209, SDHD, PAX3, SDHA, GRM3, TEC, or LARP4. Each possibility represents a separate embodiment of the invention. In some embodiments, the cancer is a skin cancer and the mutation is in a flanking region of DUX4, MALAT1, PMS2, FAT3, MLLT4, ZNF384, CDKN1A, AK2, TFEB, or WNK2. Each possibility represents a separate embodiment of the invention. In some embodiments, the cancer is a skin cancer and the mutation is a exonic synonymous mutation in MYH1, MUC4, GRIN2A, TRRAP, MUC16, PTPRT, RP1, KMT2C, NUP98, or APOB. Each possibility represents a separate embodiment of the invention. In some embodiments, the skin cancer is SKCM.

In some embodiments, the cancer is a stomach cancer and the mutation is in an intron of SET, CR1, TSC2, APOB, KMT2C, SEPT9, PARG, EP300, LRP1B, or PAX3. Each possibility represents a separate embodiment of the invention. In some embodiments, the cancer is a stomach cancer and the mutation is in a UTR of TRIM24, FAM46C, PABPC1, FOXP1, PAX3, SDHA, HMGN2, RSPO3, TEC, or HLA-A. Each possibility represents a separate embodiment of the invention. In some embodiments, the cancer is a stomach cancer and the mutation is in a flanking region of MALAT1, MLLT4, DUX4, AK2, U2, LARP4, TNFRSF14, RGPD3, BCL11A, or KLK2. Each possibility represents a separate embodiment of the invention. In some embodiments, the cancer is a stomach cancer and the mutation is a exonic synonymous mutation in MUC4, CLIP1, MUC16, COL3A1, AK2, KMT2C, SDHA, CHEK2, BCLAF1, or FAT1. Each possibility represents a separate embodiment of the invention. In some embodiments, the stomach cancer is STAD.

In some embodiments, the cancer is a thyroid cancer and the mutation is in an intron of RGS7, SET, LRP1B, CSMD3, SEPT9, CNTNAP2, PDE4D, PAX3, PARG, or ROBO2. Each possibility represents a separate embodiment of the invention. In some embodiments, the cancer is a thyroid cancer and the mutation is in a UTR of PABPC1, PAX3, CAMTA1, FAM46C, TRIM2, TBL1X, BCORL1, SDHA, CSMD3, or KDM5A. Each possibility represents a separate embodiment of the invention. In some embodiments, the cancer is a thyroid cancer and the mutation is in a flanking region of DUX4, U2, MLLT4, AK2, RGPD3, ZNF384, CRTC1, SP1, ARHGEF1, or DDX6. Each possibility represents a separate embodiment of the invention. In some embodiments, the cancer is a thyroid cancer and the mutation is a exonic synonymous mutation in MUC4, CSMD3, AK2, RANBP2, MYH1, MGA, MUC16, HLA-A, HOXA1, or KMT2C. Each possibility represents a separate embodiment of the invention. In some embodiments, the thyroid cancer is THCA.

In some embodiments, the cancer is a uterine cancer and the mutation is in an intron of PARG, TFG, CR1, PAX3, KDM6A, SETBP1, BCLAF1, SLC34A2, KMT2C, or RGPD3. Each possibility represents a separate embodiment of the invention. In some embodiments, the cancer is a uterine cancer and the mutation is in a UTR of TBL1X, BCL11B, BCR, CCND2, ABI1, PABPC1, PAX3, EXT1, RNF4, or FBLN2. Each possibility represents a separate embodiment of the invention. In some embodiments, the cancer is a uterine cancer and the mutation is in a flanking region of AK2, SP1, DUX4, YOD1, U2, RGPD3, MLLT4, PPP2R1A, FGFR2, or MECOM. Each possibility represents a separate embodiment of the invention. In some embodiments, the cancer is a uterine cancer and the mutation is a exonic synonymous mutation in MUC4, AK2, HOXA1, CLIP1, CHD4, KMT2C, USP8, HLA-A, MUC16, or RANBP2. Each possibility represents a separate embodiment of the invention. In some embodiments, the uterine cancer is UCEC.

In some embodiments, the mutation is an exonic synonymous mutation in MUC4. In some embodiments, the mutation is an exonic synonymous mutation in MUC16. In some embodiments, the mutation is a mutation in a flanking region of DUX4. In some embodiments, the mutation is a mutation in a flanking region of U2. In some embodiments, the mutation is a mutation in a flanking region of MALAT1. In some embodiments, the mutation is a mutation in a flanking region of MLLT4. In some embodiments, the mutation is an intronic mutation in SET. In some embodiments, the mutation is a UTR mutation in FAM46C.

In some embodiments, the method further comprises treating the cancer. In some embodiments, the treating comprises administering to the subject an anticancer therapy. In some embodiments, the subject is the subject that provided the sample. In some embodiments, the subject is a subject suffering from cancer. In some embodiments, the subject is a subject in need of treatment. In some embodiments, the therapy is a therapeutic agent. In some embodiments, the therapy targets the determined driver gene. In some embodiments, the therapy targets another gene in a biological pathway comprising the driver gene. In some embodiments, the gene comprises a protein produced by the gene. Biological pathways are well known as are websites and programs for determining the biological pathways comprising a gene/protein and for performing pathway analysis. Such websites and programs include but are not limited to the Reactome Pathway Database (reactome.org), KEGG pathway database, Ingenuity Pathway analysis and Gene Ontology (GO) analysis. A skilled artisan will understand that though a mutation may exist in one gene it can be indirectly targeted by therapeutics against another gene/protein in the pathway (i.e., targeting a ligand with a therapeutic against its receptor, or targeting a protein in a complex with a therapeutic against other members of the complex). Pathways relevant to the mutations can be found in Gutman et al. In particular, pathway data is provided in Supplementary Data 1 of Gutman et al., hereby incorporated by reference in its entirety.

In some embodiments, the therapy targets the determined driver mutation. In some embodiments, the therapy corrects the determined driver mutation. Methods of gene therapy and DNA correction are known in the art and any such method can be employed. Examples include CRISPR and other genome editing technologies, as well as antisense oligonucleotides (ASOs).

As used herein, the terms “administering,” “administration,” and like terms refer to any method which, in sound medical practice, delivers a composition containing an active agent to a subject in such a manner as to provide a therapeutic effect. Suitable routes of administration include oral, parenteral, subcutaneous, intravenous, intratumoral intramuscular, or intraperitoneal administration.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Methods

Data extraction: The genomic and clinical data of patients across 33 cancer types were obtained from The Cancer Genome Atlas (TCGA). Patients with multiple genomic samples and patients with no genomic samples or clinical records were excluded, leaving a total of 9,915 patients. The genomic data consists of the patients' mutation information. A genomic position is considered mutated for a patient only if its nucleic acid content differs between the patient's cancerous and healthy tissue samples.

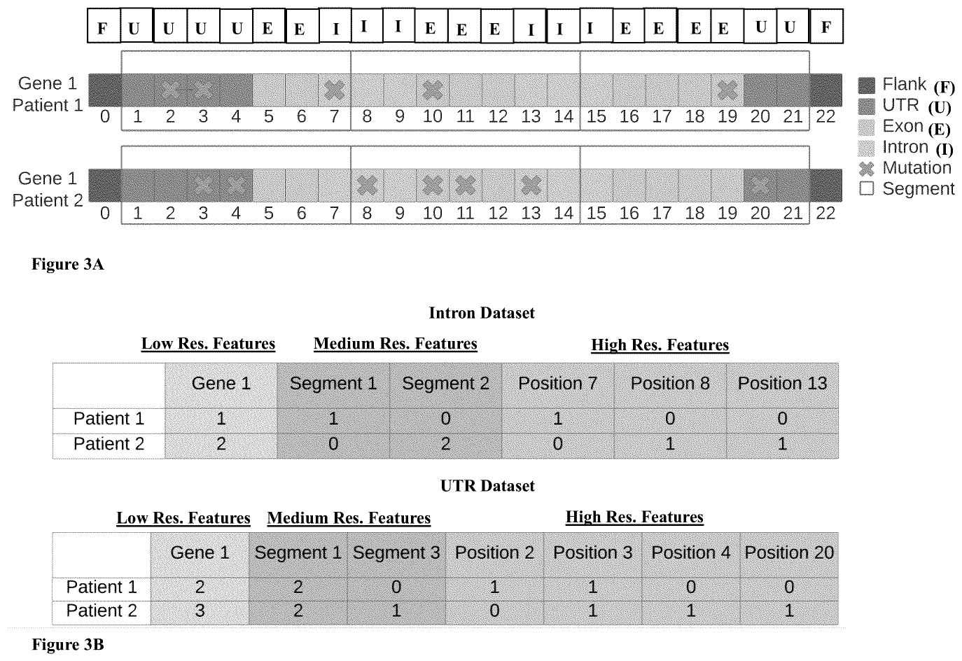

Feature engineering: Five categories of mutations were established—
1. non-silent mutations (coding sequence mutations that cause a change in the protein's amino-acid sequence)
2. synonymous mutations (coding sequence mutations that do not cause a direct change in the protein's amino-acid sequence)
3. Intronic mutations
4. UTR mutations
5. Flank mutations
   For each category, the genomic data obtained from TCGA was used to create three kinds of features, representing three levels of resolution (FIG. 3A-B): low-resolution features, medium resolution features and high-resolution features. Low-resolution features count the number of mutations that appear in an entire gene. Medium-resolution features count the number of mutations that appear in a specific segment of a gene. Each gene is assembled from the 5′UTR, introns, exons and the 3′UTR. The flanking regions are adjacent to the gene from both ends. A gene is split to 50-nucleotide long segments and the medium resolution features count the number of mutations in each segment. Two additional features count the number of mutations in the 5′ flanking regions (upstream to the gene) and in the 3′ flanking region (downstream to the gene). High-resolution features indicate whether a specific mutation occurred in a specific location in the gene (For example, an A to G SNP would be considered a different mutation than an A to C SNP, even if it had occurred in the same position). If the specific mutation occurred only for a single patient in the TCGA database, its respective feature was discarded. The features of each category were used as a separate dataset, and they were also combined in order to create the sixth dataset—the all-features dataset.

One vs. all classifiers: One vs. all classifiers were chosen to perform the classification task. As the aim was to conduct a broad, quantitative comparison between various types of mutations, we chose a classic, robust, measurable and interpretable supervised model, to lay the grounds for a fair comparison. Choosing multiple OVA classifiers, as opposed to a single multiclass classifier, enables us to easily explore which features are more closely related to which cancer type. Additionally, OVA classifiers are expected to perform better than a single multiclass classifier (as predicting a positive or negative verdict for a single cancer type is an easier task than predicting one cancer type out of 19 possibilities). Thus, if a doctor already suspects a certain cancer type, the suspicions could be validated by the relevant model with greater certainty.

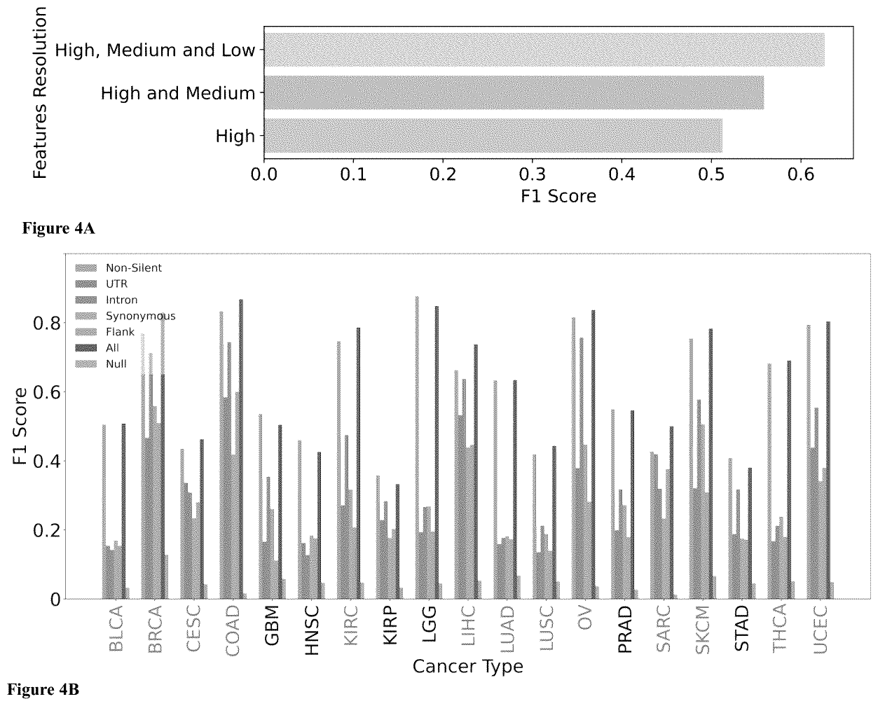

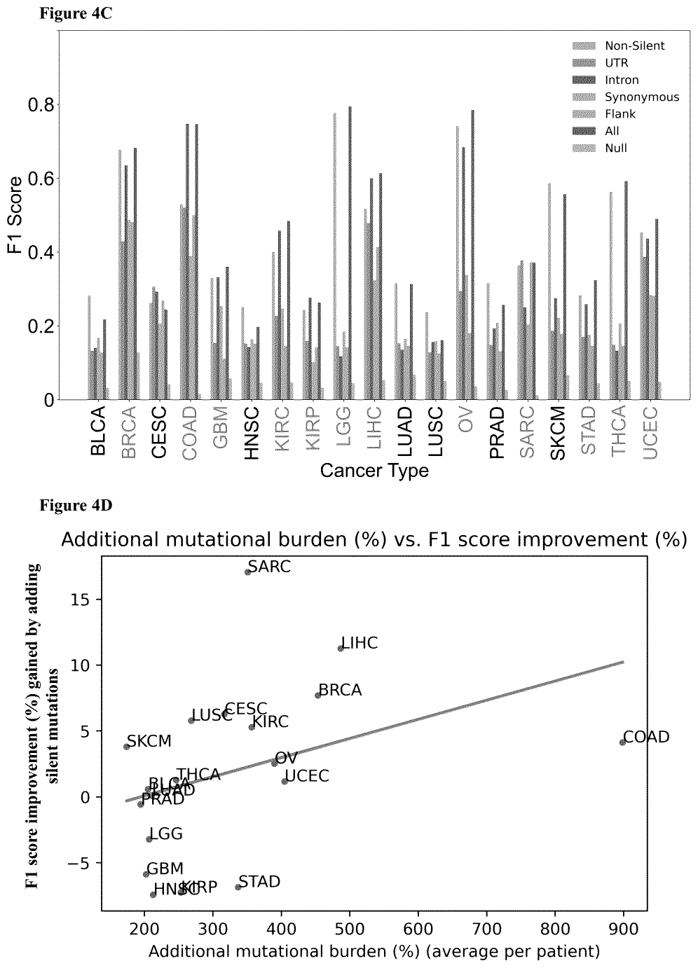

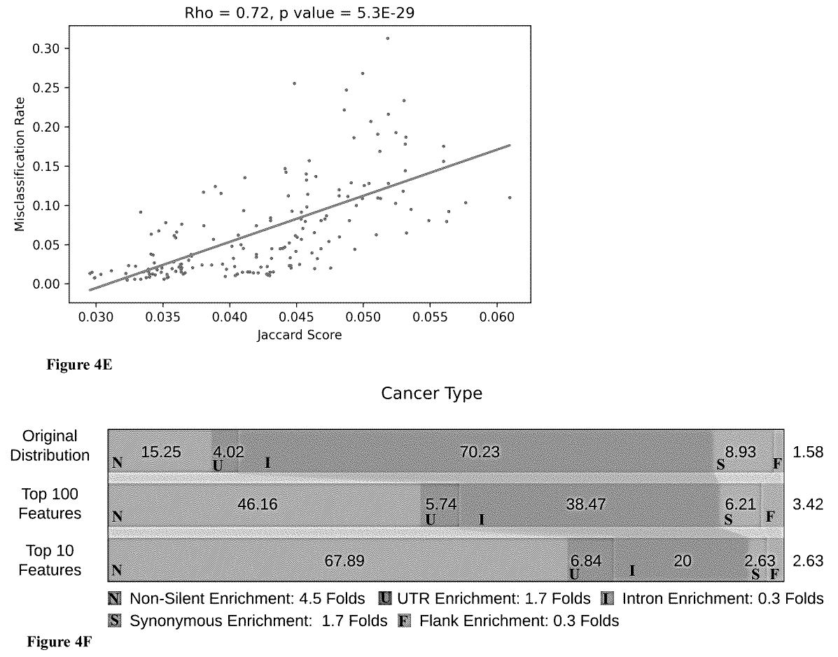

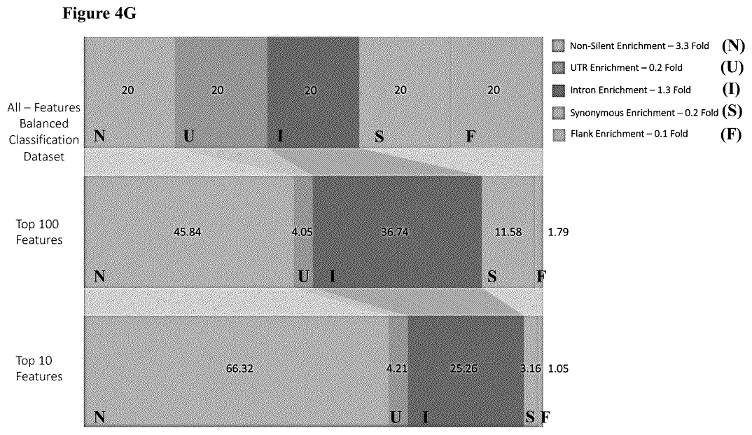

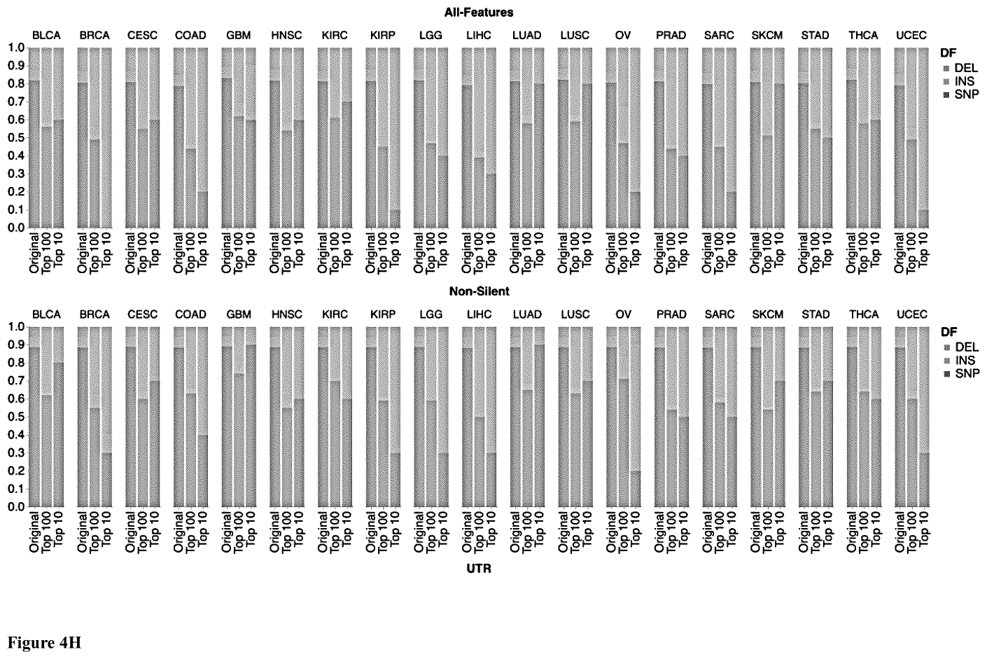

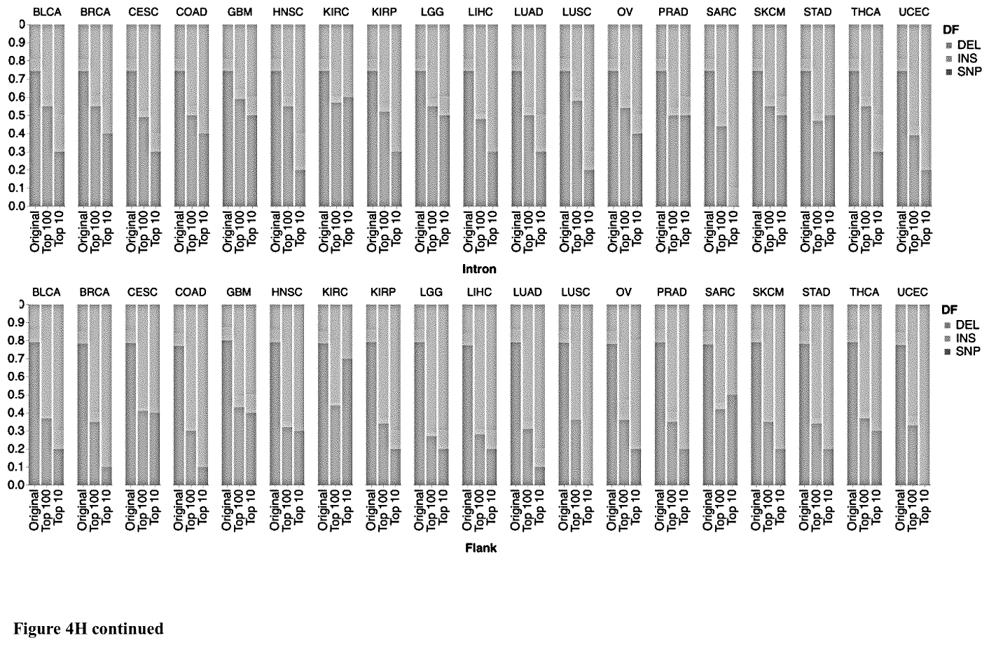

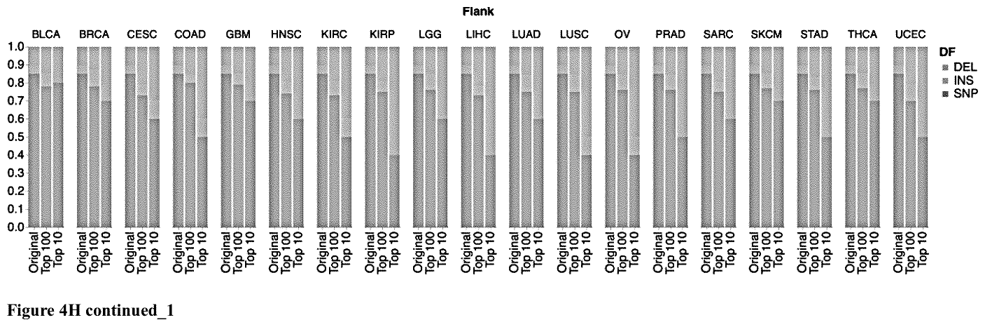

To ensure enough training examples, only cancer types with more than 200 patients were included in the analysis, resulting in 8,364 patients spanning 19 cancer types. 114 OVA classifiers were generated and trained, one for each possible combination of cancer type (19) and dataset (6). The objective of each classifier was to distinguish a single cancer type from the rest. Specifically, predicting a “Positive” or “Negative” label for a particular cancer type. The OVA classifiers were constructed using the Light GBM77 python package. For each classifier, the patients were randomly split into stratified training and testing sets (0.7/0.3 respectively) for 10 times. A null classifier was also generated using scikit-learn's Dummy Classifier for each cancer type; the null classifier randomly assigned labels to the test-set patients, only considering the label distribution of the training-set patients. The classifiers' performance was evaluated with Accuracy, Recall, Precision and F1 scores (FIG. 4B). Performances were averaged across the 10 splits. Precision is the fraction of correctly identified positive patients out of all patients that were identified as positive by the model. Recall is the fraction of correctly identified positive patients out of all the patients that are truly positive for the disease. The F1 score is a harmonic mean of precision and recall, taking both measures into account:

$$f 1 = 2 * \frac{P * R}{P + R} \qquad ( 1 )$$

Where P is Precision and R is Recall. The F1 score ranges from zero to one, one indicating perfect Precision and Recall scores and zero indicating that either the Precision or Recall are also zero.

Gene ranking: Each classifier provides a feature ranking. First, features with zero importance were discarded. Then, a gene ranking was obtained by assigning the features (that can be mutations, segments or entire genes) to the gene they are related to while keeping the original order. Finally, only the highest rank of each gene was kept. The most important gene is ranked “0” and as the numbers increase the importance decreases.

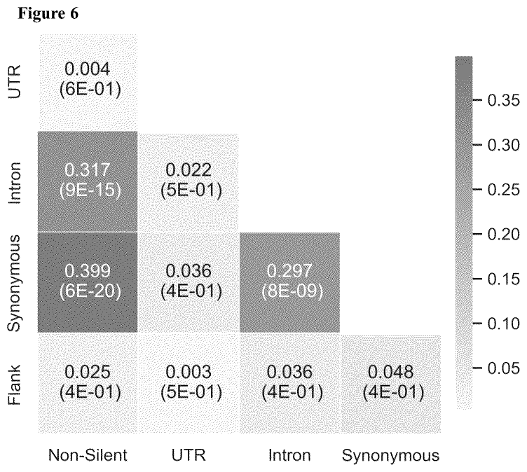

Spearman correlation between gene rankings: Spearman correlations were conducted between gene rankings of pairs of classifiers detecting the same cancer type (FIG. 6). For every cancer type:
1. The all-features classifier was excluded.
2. For each of the single-mutation-type classifiers, a gene ranking list was created as described above.
3. Every combination of two classifiers was examined; genes that were not in the intersection of both gene ranking lists were discarded. Spearman correlation was calculated between the revised gene ranking lists.
   The results were averaged across the 19 cancer types.

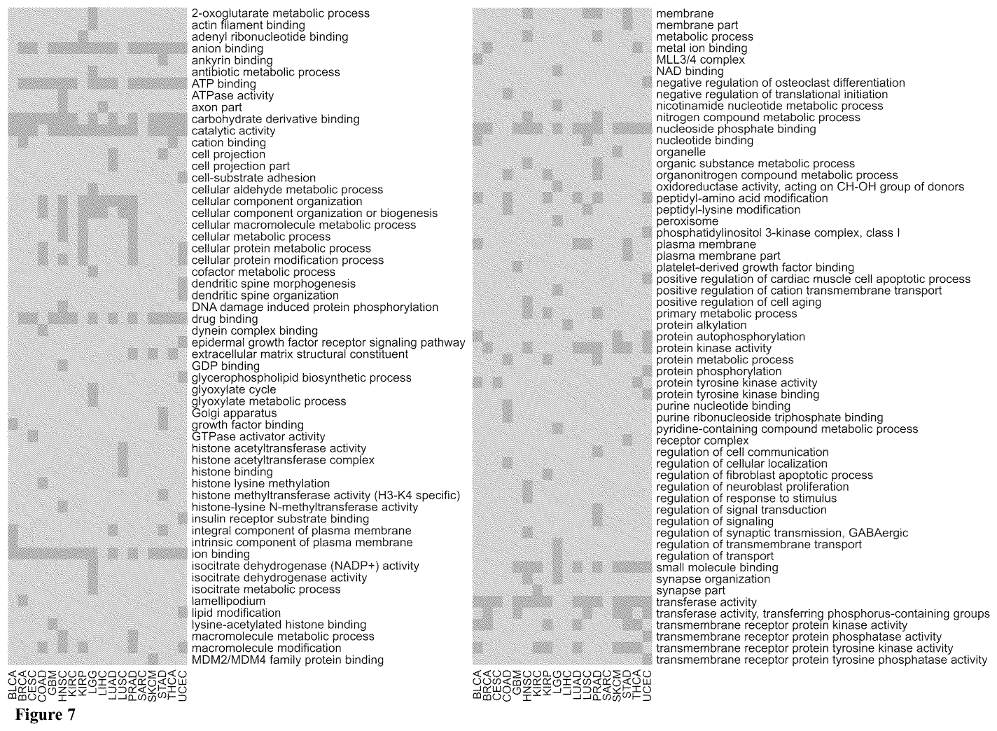

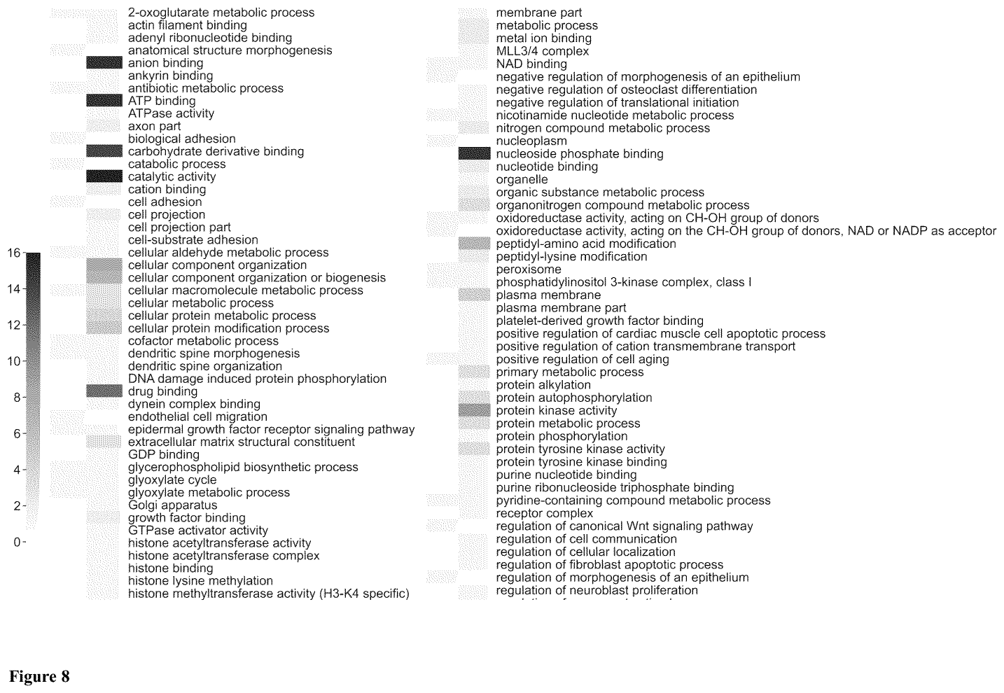

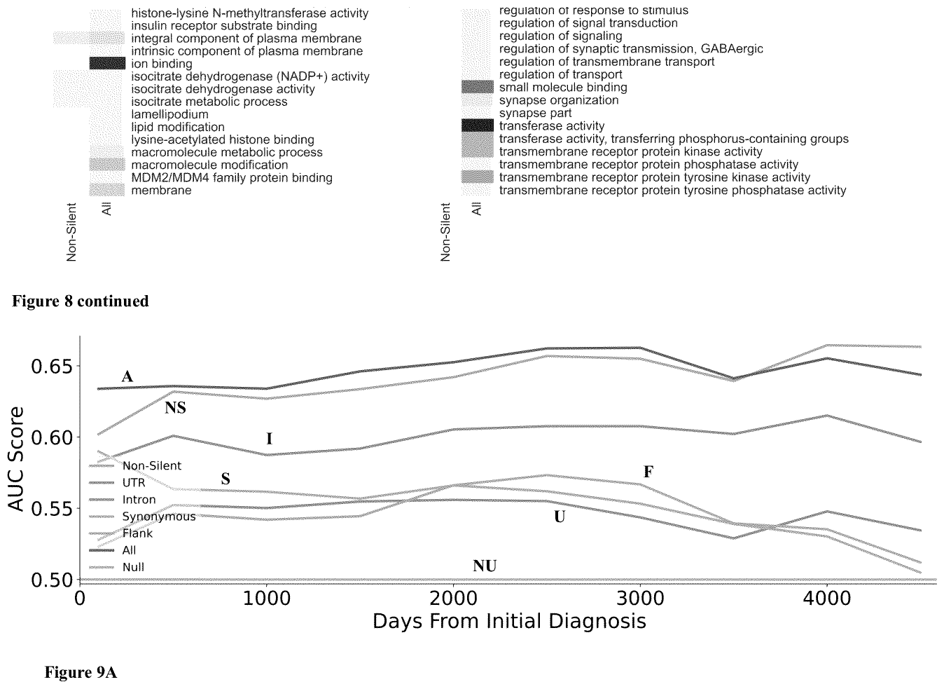

Gene ontology enrichment: Enriched GO terms (molecular functions, biological processes and cellular components) were detected for the 19 cancer types using the gene rankings obtained from the different models. For every combination of cancer type and model:
1. The gene ranking list was created as described above.
2. The gene ranking list was used as input to the GOrilla tool 44, 45. The tool used maximum Hyper Geometric (mHG) statistics in order to report GO terms that are enriched in the top of the list compared to the rest of the list. The threshold for splitting the genes list to “top” and “rest” is dynamic and was chosen for each GO term individually by the tool.
3. The yielded terms are enriched with a p-value smaller than 0.001 and have passed an FDR correction of 0.05.
4. The yielded terms were used as input to the REVIGO tool, which removed terms with a semantic similarity score higher than 0.7. The similarity measure used was “SimRel”.
   The enriched GO terms detected for the 19 cancer types when using the all-features gene ranking are detailed in FIG. 7. A comparison between the GO terms that are detected when using the all-features gene ranking or the non-silent gene-ranking is seen in FIG. 8.

Pathway enrichment: Enriched pathways were detected for the 19 cancer types using the gene rankings obtained from the different models. For every combination of cancer type and model:
1. The gene ranking list was created as described above.
2. The highest ranked 50 genes in the list was used as input to the REACTOME pathway enrichment analysis tool. The number of genes was chosen considering both statistical power and the total length of the gene list.
3. The REACTOME yielded enriched pathways. An enriched pathway is a pathway for which the number of genes in the provided list that is associated to it is larger than expected by chance, considering both the total amount of genes known to be associated with the pathway and the number of gene in our list. The yielded pathways obtained an FDR value that is smaller than 0.01.

Mutational burden: The analysis presented in FIG. 4D was conducted to evaluate whether the improvement in classification that was gained from adding silent features to non-silent features was obtained because of the additional mutational burden. For each cancer type:
1. The percent of improvement gained from adding silent features was calculated as shown in equation (2):

$$F {1}_{i m p r o v e m e n t} = \frac{F {1}_{a l l - f e a t u r e s} - F {1}_{n o n - s i l e n t}}{F {1}_{n o n - s i l e n t}} * 100 \qquad ( 2 )$$

where F1_{all-features} is the F1 score of the all-features model of the current cancer type.
2. The percent of mutational burden gained from adding silent features (an average across patients) was calculated as shown in equation (3):

$$M {B}_{i n c r e a s e} = \frac{{\sum }_{i = 1}^{n} ( \frac{M {B}_{i , all - features} - {MB}_{i , non - silent}}{M {B}_{i , non - s i l e n t}} * 100 )}{n} \text{.3} \qquad ( 3 )$$

Where MB_{i,all-features} is the mutational burden (number of mutations) that the i\hat{ }th patient in the all-features dataset has and n is the number of patients of the current cancer type.

Then the correlation between F1ᵢₘₚᵣₒᵥₑₘₑₙₜ and MB_{increase} among the cancer types was examined.

Spearman correlation between Jaccard similarity scores and misclassification rates: A Spearman correlation was conducted in order to evaluate the influence of genetic profile similarity on misclassification rates among pairs of cancer types. For this analysis binary versions of the features were used, meaning that rather than indicating how many mutations occur in genes and segments the features indicate whether any mutations had occurred or not (high resolution features were originally binary and thus do not change). Calculating the Jaccard similarity scores for every pair of cancer types was performed in the following manner:
1. 100 patients were randomly selected from each type, forming two equally sized groups of patients (groups A and B).
2. A Jaccard score was calculated for every patient in the group A with every patient in group B. The average score was considered the Jaccard score between the groups. The calculation was performed as shown in equation (4):

$${J}_{A , B} = \frac{{\sum }_{a = 1}^{100} {\sum }_{b = 1}^{100} \frac{\vert {F}_{a} ∩ {F}_{b} \vert}{\vert {F}_{a} \vert + \vert {F}_{b} \vert - \vert {F}_{a} ∩ {F}_{b} \vert}}{100 * 100} \qquad ( 4 )$$

Where Fₐ is the binary feature set of patient a from group A and F_{b} is the binary feature set of patient b from group B. |Fₐ| is the number of features equal to “1” for patient a from group A (indicating all positions, segments and entire genes that were mutated). J_{A,B} is the average Jaccard similarity score between group A and group B.
3. The random sampling process was repeated 5 times. The final Jaccard score for a pair of cancer types was the average of the five repetitions.

Calculating the mistake rate for every pair of cancer types was performed in the following manner:
1. 250 patients were randomly selected from each type (groups A and B).
2. The patients were stratified split to train and test sets (the training-set contained 70% of patients from each cancer types).
3. An OVA model was fit on the training-set patients.
4. The model was used to classify the test-set patients to one of the two cancer types.
5. The misclassification rate between the groups was calculated as shown in equation (5):

$${M}_{A , B} = \frac{\vert AB \vert + \vert BA \vert}{\vert AA \vert + \vert BB \vert + \vert AB \vert + \vert BA \vert} \qquad ( 5 )$$

Where |AB| is the number of group-A-patients that were classified as group-B-patients. M_{A,B} is the misclassification rate between groups A and B.
6. The random sampling process was repeated 10 times. The misclassification rate between the pair of cancer types was the average of the 10 repetitions.

Balanced datasets: To evaluate whether the results are significantly influenced by the imbalance between the mutation categories, balanced datasets were created for the two analyses depicted in FIG. 4B and FIG. 4F. To maintain the balance, only high-resolution features were used in these datasets. Six same-size datasets were needed for the balanced version of FIG. 4B. For every cancer type:
1. The patients were split to two equally sized groups. The first for feature selection and creation of the balanced datasets and the second for training models on the balanced datasets and evaluating the results.
2. For creating the balanced datasets six OVA models (one per dataset) were trained using the first group of patients and all their features were ranked. For every model, the highest ranked 8,296 features were chosen as the new dataset. This step resulted in six balanced datasets per cancer type, each containing 8,296 features. (The number of features was derived from the number of features in the smallest category, the flanking region mutations).
3. The six OVA models (one per dataset) were trained using the second group of patients and the balanced datasets. The models were trained for 10 rounds, whereby on each round a stratified random 0.7/0.3 split was performed. The performance was evaluated using the same measures as the imbalanced version of this analysis.

For the balanced version of FIG. 4F an all-features dataset with an internal balance between mutation types was needed. For every cancer type, the 8,296 features that were chosen from each of the five mutation categories were combined in order to create the internally balanced all-features dataset. Then, an OVA model was trained using the balanced dataset and the second group of patients. The model was trained for 10 rounds, whereby on each round a stratified random 0.7/0.3 split was performed. The mutation-types distribution among the top 10 and top 100 features chosen by the classifiers were averaged across cancer types.

Random survival forest models: A random survival forest model is an adaptation of the random forest model, modified to perform survival estimations. Its performance is comparable and sometimes better than classic survival models such as Cox regression. The RSF is a non-parametric data-driven approach that is independent of model assumptions. It was chosen for our survival estimation task because it is known to perform well specifically with high dimensional datasets, compared to traditional approaches (for example, Cox regression relies on several assumptions that are usually violated in high-dimensional datasets).

Patients spanning all 33 cancer types were included in this analysis (as this is not a classification task and there was no need to remove small cohorts). Patients with no available information after the date of diagnosis and patients who passed away less than 20 days after their diagnosis were not included. Overall, 9,551 patients were incorporated in the analysis. The patients are treated as a single cohort and the model is oblivious of their cancer type. Unlike the classification task, this analysis is not performed separately for each cancer types because it requires more data (e.g., while the OVA model that diagnose BRCA trains on both BRCA-positive and BRCA-negative patients, the RSF model that estimates the survival of BRCA patients only trains on BRCA-positive patients while aiming at estimating an entire survival curve, and thus has a much smaller patient cohort to train on). The vital status (alive or deceased) and appropriate time stamp were extracted from the clinical data and used as labels. A subset of features was chosen for each mutation category-all low-resolution features and 5,000 high-resolution features. The high-resolution features were selected based on mutation prevalence in TCGA; the features corresponding to the 5,000 most prevalent mutations were selected.

A model was generated and trained for each one of the six datasets (non-silent, UTR, intron, synonymous, flank and all-features). The objective of a model was to predict the probability of a patient to survive on a given time after its initial cancer diagnosis. The models were constructed using the Pysurvival Python package. 60 trees were grown with a maximal depth of 32 splits. At each split, Kaplan Meier estimators and the log-rank test were used to find the feature is the best separator. For each model, the patients were randomly split into training and testing sets (0.7/0.3 respectively). The model was trained using the training set patients and then tested on the patients of the test set, which the model has never encountered before. To avoid biases introduced by a specific split, the process was repeated five times and the survival probability estimation is the average of the 5 repetitions.

The models' performances on the test set patients were evaluated using the Area Under the Curve (AUC) score for various times (100, 500, 1000, 1500, 2000, 2500, 3000, 3500, 4000 and 4500 days) after the initial cancer diagnosis. After 4500 days the data is scarce, as most patients have stopped attending follow-ups or have passed away. Thus, the analysis was terminated at this point.

Predicting the regulatory effects of highly ranked features: Predictive models were used to assess the influence of mutations spanned by the top ten ranked features of each cancer type (whether they are of low, medium or high resolution) on splice sites (using SpliceAI), miRNA binding sites (cnnMirTarget), mRNA expression levels (using Xpresso), polyadenylation (using SANPolyA), 3D folding (using Akita) and several protein-mRNA binding sites (using DeepCLIP).

### Data Availability:

The clinical data and simple nucleotide variation (SNV) data that was used herein was generated by The Cancer Genome Atlas (cancer.gov/tcga) and can be downloaded from the genomic data commons. Specifically, data of the following projects were used for the classification task: TCGA-BRCA (n=1023), TCGA-UCEC (n=536), TCGA-HNSC (n=506), TCGA-LGG (n=496), TCGA-PRAD (n=493), TCGA-LUAD (n=488), TCGA-THCA (n=486), TCGA-SKCM (n=468), TCGA-STAD (n=441), TCGA-LUSC (n=435), TCGA-BLCA (n=408), TCGA-COAD (n=402), TCGA-LIHC (n=373), TCGA-OV (n=372), TCGA-KIRC (n=308), TCGA-CESC (n=303), TCGA-GBM (n=292), TCGA-KIRP (n=283) and TCGA-SARC (n=251). Data of these aforementioned projects and the following projects were used for the survival estimation task: TCGA-ESCA (n=183), TCGA-PAAD (n=182), TCGA-PCPG (n=175), TCGA-READ (n=151), TCGA-THYM (n=123), TCGA-ACC (n=92), TCGA-MESO (n=83), TCGA-UVM (n=80), TCGA-KICH (n=66), TCGA-UCS (n=57), TCGA-DLBC (n=48) and TCGA-CHOL (n=45). The data was downloaded from the Genomic Data Commons (portal.gdc.cancer.gov/) in December 2018.

### Example 1: Data Processing and Feature Engineering

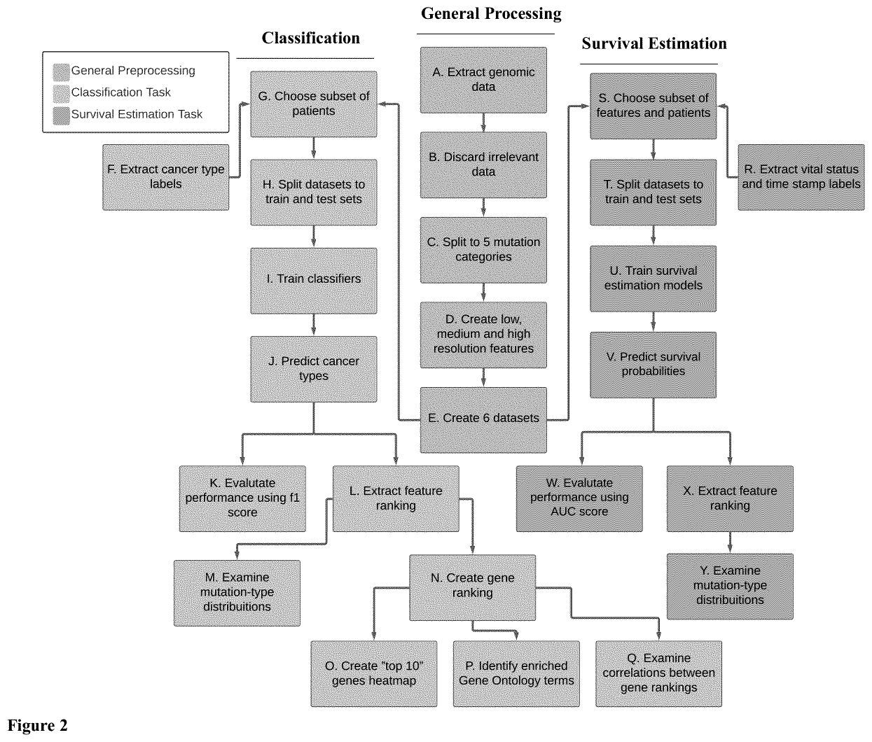

Genomic and clinical data of 9,915 patients across 33 cancer types were obtained from The Cancer Genome Atlas (TCGA). Data characteristics are described in FIGS. 1A-D. The genomic data consisted of detailed information about the patients' DNA mutations while the clinical data held personal information such as patients' vital status. These data were used to perform two tasks: patients' cancer type classification and survival estimation. A full flow chart of the study is provided in FIG. 2.

As FIG. 2 indicates, the genomic data was split into five categories. One category holds all non-silent mutations (amino-acid-altering exonic mutations). The other four categories consist of silent mutations from different regions within and adjacent to the genes; synonymous mutations (exonic mutations that do not directly affect the amino acid sequence), mutations in introns, mutations in UTRs and mutations in flanking regions (5000 nt upstream and 5000 nt downstream of the gene). It is important to note that a genomic position is considered mutated for a patient only if its nucleic acid content differs between the patient's cancerous and healthy tissue samples.

In the next preprocessing step, for each category, the initial data was used to create three kinds of features (FIG. 3A-B), representing different resolutions—
1. Low resolution features—indicating the number of mutations each patient had in an entire gene.
2. Medium resolution features—indicating the number of mutations each patient had in a 50-nucleotide-long gene segment.
3. High resolution features—binary features indicating whether a specific mutation occurred or not, for each patient.

Analyzing features from multiple resolution levels improves the models' results (FIG. 4A, Table 1) and could also identify specific mutations, regulatory regions and entire genes that are related to cancer fitness.

TABLE 1
F1 score improvement gained from adding lower resolution features.
The F1 score improvement per cancer type that was achieved by adding
medium resolution features and then low-resolution features is provided.
The results were obtained using the all-features models.

| | | High and | High, Medium | Total |
|---|---|---|---|---|
| Cancer | High | Medium | and Low | Improvement |
| Type | Resolution | Resolutions | Resolutions | (%) |
| BRCA | 0.72 | 0.76 | 0.83 | 15.27 |
| UCEC | 0.58 | 0.73 | 0.8 | 37.99 |
| HNSC | 0.31 | 0.34 | 0.43 | 38.59 |
| LGG | 0.8 | 0.85 | 0.85 | 5.79 |
| PRAD | 0.34 | 0.43 | 0.55 | 59.18 |
| LUAD | 0.39 | 0.52 | 0.63 | 61.38 |
| THCA | 0.63 | 0.64 | 0.69 | 10.22 |
| SKCM | 0.66 | 0.68 | 0.78 | 17.79 |
| STAD | 0.32 | 0.35 | 0.38 | 20.48 |
| LUSC | 0.28 | 0.29 | 0.44 | 60.61 |
| BLCA | 0.32 | 0.36 | 0.51 | 57.63 |
| COAD | 0.78 | 0.83 | 0.87 | 10.84 |
| LIHC | 0.64 | 0.69 | 0.74 | 15.92 |
| OV | 0.81 | 0.81 | 0.84 | 2.7 |
| KIRC | 0.57 | 0.68 | 0.79 | 38.37 |
| CESC | 0.37 | 0.41 | 0.46 | 25.46 |
| GBM | 0.46 | 0.45 | 0.5 | 10.09 |
| KIRP | 0.32 | 0.34 | 0.33 | 3.93 |
| SARC | 0.44 | 0.47 | 0.5 | 12.67 |

The features created for each of the five categories were used as five separate datasets (referred to as single-mutation-type datasets). A sixth dataset that combines features of all mutation types (referred to as all-features dataset) was also created. The six datasets were used to perform cancer type diagnosis and patient survival estimation. Evaluating the performance of models trained on the six datasets enables us to compare the predictive ability of features derived from silent and non-silent mutations (referred to as silent features and non-silent features).

### Example 2: For all Cancer Types, the Silent Features Improved Cancer Classification in Comparison to the Null Model

In the cancer type classification task, only cancer types with more than 200 patients were included (a total of 19 types). A one-vs-all (OVA), supervised learning model was created for every pair of cancer type and dataset (see Methods). Specifically, each model deployed the features in the dataset in order to predict whether patients suffered from the specific cancer type (classified as “Positive”) or suffered from any of the other types (classified as “Negative”, since the model predicts only the existence of the specific cancer).

As mentioned above, combining features from three levels of resolutions led to the best performance of cancer type classification. FIG. 4B depicts the F1 scores (see equation (1) for the definition of the F1 score) obtained by the OVA models by using features from all levels of resolutions. The worst performing model, which used flanking-region features in order to diagnose Glioblastoma (GBM), was 1.9 folds better than the comparable null model (see Methods for details about the null models). The best performing model that used silent features was the intron model for diagnosing Ovarian Serous Cystadenocarcinoma (OV), and its F1 score was 20 folds higher than the comparable null model. Even though the non-silent models generally achieved better results than silent models, for several cancer types the performances were substantially similar. For example, for detection of Breast Invasive Carcinoma (BRCA), Liver Hepatocellular Carcinoma (LIHC) and OV the performance difference between the non-silent model and the intron model was less than 10%. For Sarcoma (SARC) diagnosis, the non-silent model outperformed the UTR model by a mere 2%, and the flank model was exceeded by only 12%. In addition, the all-features models, which used both silent and non-silent features, obtained higher F1 scores than the non-silent models for 13 out of the 19 cancer types (denoted in grey in FIG. 4B) and for the other cancer types, the performances were comparable. Similar results were observed when all silent mutations were combined without the non-synonymous mutations (data not shown).

To control for the number of features, the same analysis was conducted using balanced datasets as well (see Methods) and the results, shown in FIG. 4C, accentuate the high diagnostic ability of silent mutations. In the balanced version, the Intron model outperformed the non-silent model for six cancer types (CESC, COAD, GBM, KIRC, KIRP, and LIHC) and the UTR and flank models were superior to the non-silent model for two cancer types (CESC and SARC for both). Quite similarly to the unbalanced datasets, combining silent and non-silent mutations rather than solely using the latter improved classification results for 12 out of 19 cancer types (keeping in mind that the all-features dataset had the same number of features as the non-silent dataset in this analysis). All these findings support the hypothesis that silent mutations do affect cancer mechanisms and hold additional predictive information that could not be obtained from non-silent mutations alone. Another confounder that could have influenced the classification results is the total mutational burden. To ensure that the improvement gained from adding silent features to non-silent features is not mainly due to the increase in the total mutational burden that occurs because of the addition, we examined how the increase in total mutational burden is correlated with the improvement in the F1 scores of the different cancer types (FIG. 4D). Results demonstrate a Pearson correlation of R=0.38 (p=0.1), indicating that only 14% of the change in the F1 score could be explained by the increase in mutational burden. So, even though the mutational burden does impact the results of classification, it is not the leading factor. Similar results were observed when all silent mutations were combined without the non-synonymous mutations.

Another interesting phenomenon demonstrated in FIG. 4B is the considerable differences in the models' ability to diagnose different cancer types. While the majority of the BRCA, LGG (Lower Grade Glioma) or COAD (Colon Adenocarcinoma) patients were correctly diagnosed (by at least one model), KIRP (Kidney Renal Papillary Cell Carcinoma) and STAD (Stomach Adenocarcinoma) patients were often poorly diagnosed. To explore the origin of this difference, the similarity between genetic profiles of the different cancer types were examined and whether cancers with higher genetic similarity have higher misclassification rates was assessed. For every pair of cancer types, the correlation between their Jaccard similarity score and their misclassification rate was inspected (see Methods). The results (FIG. 4E) indicate a Spearman correlation coefficient of 0.72 (p value<10\hat{ }(−28)), suggesting the similarity between genetic profiles of patients of different cancers is indeed a major cause for misclassifications. However, this is not the only cause as it only explains ˜52% of the variance in their misclassification rate. Another factor that could lead to misclassifications is high mutation heterogeneity among patients of the same cancer type.

### Example 3: Silent Features Comprise 32% of the 10 Most Predictive Features for Cancer Classification, on Average, Across Cancer Types

Each OVA model provides an importance ranking for all its features. Examining the ranking of silent features among all features is another way to evaluate their predictive power. Reviewing the feature importance ranking produced by the all-features models, silent features comprised nearly half of the top ranked 100 features and a third of the top ranked 10 features (chosen from hundreds of thousands of features), when averaged across cancer types (FIG. 4F). However, the ranking of silent features varied substantially between cancer types (Tables 2 and 3); while there were only non-silent features in the top 10 features of Lung Adenocarcinoma (LUAD), silent features constituted eight out of the top 10 features of Cervical Squamous Cell Carcinoma (CESC). Altogether, 18 out of the 19 cancer types had at least one silent feature in their top 10 features list, demonstrating their high significance. The analysis was repeated with balanced datasets and the results were similar (FIG. 4G).

TABLE 2
Feature type distribution among the top 100 ranked
features for each cancer type. Feature rankings
were obtained from the all-features models.

| Cancer\Mutation | Non-Silent | Intron | UTR | Flank | Synonymous |
|---|---|---|---|---|---|
| Category | (%) | (%) | (%) | (%) | (%) |
| BLCA | 64 | 33 | 0 | 2 | 1 |
| BRCA | 51 | 36 | 1 | 5 | 7 |
| CESC | 27 | 37 | 21 | 8 | 7 |
| COAD | 34 | 42 | 11 | 9 | 4 |
| GBM | 58 | 38 | 0 | 0 | 4 |
| HNSC | 53 | 39 | 1 | 2 | 5 |
| KIRC | 49 | 38 | 3 | 4 | 6 |
| KIRP | 53 | 37 | 5 | 4 | 1 |
| LGG | 48 | 47 | 0 | 0 | 5 |
| LIHC | 25 | 43 | 19 | 8 | 5 |
| LUAD | 48 | 40 | 0 | 2 | 10 |
| LUSC | 45 | 34 | 2 | 3 | 16 |
| OV | 44 | 45 | 6 | 2 | 3 |
| PRAD | 52 | 43 | 0 | 2 | 3 |
| SARC | 23 | 34 | 31 | 6 | 6 |
| SKCM | 48 | 36 | 0 | 2 | 14 |
| STAD | 53 | 37 | 2 | 0 | 8 |
| THCA | 59 | 29 | 0 | 1 | 11 |
| UCEC | 43 | 43 | 7 | 5 | 2 |

TABLE 3
Feature type distribution among the top 10 ranked
features for each cancer type. Feature rankings
were obtained from the all-features models.

| Cancer\Mutation | Non-Silent | Intron | UTR | Flank | Synonymous |
|---|---|---|---|---|---|
| Category | (%) | (%) | (%) | (%) | (%) |
| BLCA | 80 | 10 | 0 | 10 | 0 |
| BRCA | 60 | 30 | 0 | 10 | 0 |
| CESC | 20 | 10 | 50 | 10 | 10 |
| COAD | 40 | 10 | 0 | 10 | 0 |
| GBM | 70 | 30 | 0 | 0 | 0 |
| HNSC | 80 | 10 | 0 | 10 | 0 |
| KIRC | 60 | 40 | 0 | 0 | 0 |
| KIRP | 60 | 40 | 0 | 0 | 0 |
| LGG | 80 | 20 | 0 | 0 | 0 |
| LIHC | 30 | 40 | 20 | 0 | 10 |
| LUAD | 100 | 0 | 0 | 0 | 0 |
| LUSC | 90 | 10 | 0 | 0 | 0 |
| OV | 80 | 20 | 0 | 0 | 0 |
| PRAD | 80 | 20 | 0 | 0 | 0 |
| SARC | 30 | 10 | 60 | 0 | 0 |
| SKCM | 90 | 0 | 0 | 0 | 10 |
| STAD | 80 | 20 | 0 | 0 | 0 |
| THCA | 80 | 10 | 0 | 0 | 10 |
| UCEC | 80 | 10 | 0 | 0 | 10 |

When evaluating the influence of the polymorphism type (whether a mutation is an insertion, a deletion or an SNP) on the importance ranking, it was seen that the presence of deletions in the highly ranked features was notably higher than their presence in the initial datasets (FIG. 4H). In fact, their prevalence in the top 10 features was 2.9-6.8 folds higher than their prevalence in the initial datasets (varying between the different models). The presence of SNPs and insertions in the highly ranked features was lower than their presence in the initial datasets, with the exception of the UTR dataset, for which the insertions were 1.3 folds more common in the top 10 features lists than in the initial datasets, on average across cancer types.

### Example 4: A Gene's Predictive Power for Cancer Type Classification Varies Drastically when Mutated by Different Types of Mutations

Table 4 lists the 10 most predictive features of three of the 19 cancer types, as chosen by the all-features models. As seen in Table 4, some genes appeared in the top 10 ranked genes for multiple cancer types. MUC4 was in the top 10 list for 16 out of the 19 cancer types and TP53 was on 11 lists, suggesting these genes could play an essential role in cancer mechanisms. Interestingly, MUC4 was predictive of many cancer types when it had either non-silent mutations or synonymous mutations. This last finding raises the following fundamental question: is the mutation type a determining factor in a gene's ability to predict a cancer type? Or perhaps different kinds of alterations in various regions of the same gene would cause a similar loss or gain of function, leading to the same outcomes on cancer development?

TABLE 4
Examples of the top 10 ranked features for classifying various cancer
types. The top 10 feature rankings for CESC, LIHC and THCA are shown.
For each feature, the table holds its name, mutation type, its importance
for classifying the specific cancer type and the gene to which it is
related to. The rankings were obtained from the all-features models.

| Cancer | | | Feature | | |
|---|---|---|---|---|---|
| Type | Rank | Feature | Type | Importance | Gene |
| CESC | 0 | MUC4 Non_Silent | Non-Silent | 0.16 | MUC4 |
| | 1 | TP53 Non_Silent | Non-Silent | 0.06 | TP53 |
| | 2 | PABPC1 UTR | UTR | 0.04 | PABPC1 |
| | 3 | BCR UTR | UTR | 0.03 | BCR |
| | 4 | NF1 5602.0 UTR | UTR | 0.01 | NF1 |
| | 5 | RGPD3 0.0 Flank | Flank | 0.01 | RGPD3 |
| | 6 | CSF1 UTR | UTR | 0.01 | CSF1 |
| | 7 | MUC4 Synonymous | Synonymous | 0.01 | MUC4 |
| | 8 | SRGAP3 UTR | UTR | 0.01 | SRGAP3 |
| | 9 | CARD11 793.0 | Intron | 0.01 | CARD11 |
| | | Intron | | | |
| LIHC | 0 | MUC4 Non_Silent | Non-Silent | 0.25 | MUC4 |
| | 1 | SET 210.0 Intron | Intron | 0.08 | SET |
| | 2 | PIK3CA | Non-Silent | 0.03 | PIK3CA |
| | | Non_Silent | | | |
| | 3 | ALB Intron | Intron | 0.02 | ALB |
| | 4 | 240343-240343-chr5- | Intron | 0.02 | SDHA |
| | | Intron-DEL-T-T-- | | | |
| | 5 | APC Non_Silent | Non-Silent | 0.01 | APC |
| | 6 | FAM46C UTR | UTR | 0.01 | FAM46C |
| | 7 | SRGAP3 UTR | UTR | 0.01 | SRGAP3 |
| | 8 | MUC4 Synonymous | Synonymous | 0.01 | MUC4 |
| | 9 | SEPT9 3283.0 | Intron | 0.01 | 1-Sep |
| | | Intron | | | |
| THCA | 0 | 140753336- | Non-Silent | 0.18 | BRAF |
| | | 140753336-chr7- | | | |
| | | Missense_Mutation- | | | |
| | | SNP-A-A-T | | | |
| | 1 | BRAF 378.0 | Non-Silent | 0.13 | BRAF |
| | | Non_Silent | | | |
| | 2 | TP53 Non_Silent | Non-Silent | 0.07 | TP53 |
| | 3 | MUC4 Non_Silent | Non-Silent | 0.06 | MUC4 |
| | 4 | NRAS 189.0 | Non-Silent | 0.02 | NRAS |
| | | Non_Silent | | | |
| | 5 | MUC4 Silent | Synonymous | 0.02 | MUC4 |
| | 6 | 533874-533874-chr11- | Non-Silent | 0.01 | HRAS |
| | | Missense_Mutation- | | | |
| | | SNP-T-T-C | | | |
| | 7 | BRAF Non_Silent | Non-Silent | 0.01 | BRAF |
| | 8 | TP53 26.0 | Non-Silent | 0.01 | TP53 |
| | | Non_Silent | | | |
| | 9 | LRP1B Intron | Intron | 0.01 | LRP1B |

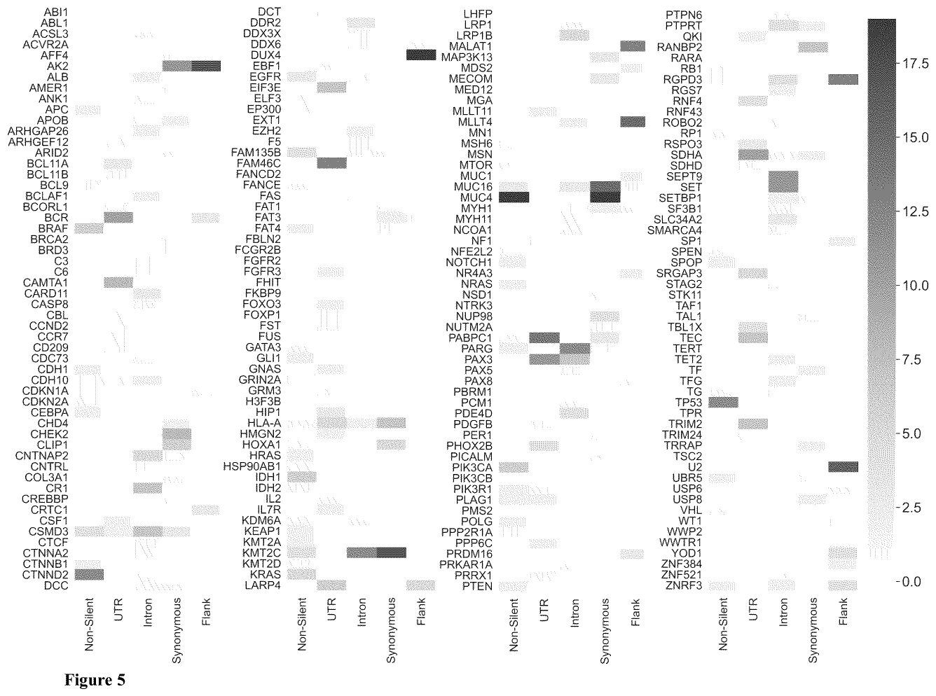

To try and answer this question, the top 10 features list from every single-mutation-type OVA model was examined (the all-features models were excluded from this analysis). For each cancer, a top 10 genes list was derived from the top 10 features list (see Methods). FIG. 5 depicts a heatmap, presenting the number of top 10 genes lists a gene has appeared in (19 meaning the gene appeared in the top 10 genes lists of all cancer types, and zero meaning it had appeared in none). As seen in FIG. 5, the number of appearances a gene has in the top 10 lists changes dramatically when it is mutated by mutations of different types. For example, the aforementioned MUC4 gene appears in all 19 lists when it is mutated by non-silent mutations or synonymous mutations, but when it is mutated in the UTR, introns or flanks it loses its predictive significance and does not appear in any of the lists. In fact, it is evident that most genes are highly predictive of multiple cancer types only when mutated by a specific mutation type. For example, MUC16 is highly predictive of 15 cancer types, but only if its mutations are synonymous. Altogether, it is evident that the mutation type does influence the predicative power a gene has on cancer diagnosis. Nonetheless, it can also be seen that for some genes, such as AK2 or KTM2C, more than a single mutation type leads to high productivity of multiple cancers. So, even though it has been established that not all mutations cause the same effect, perhaps some lead to more similar consequences than others.

The top (by importance) 10 genes bearing silent mutations for each of the cancers investigated herein are provided in Table 5. A full list of all mutations can be found in Gutman et al., 2021, “Estimating the predictive power of silent mutations on cancer classification and prognosis”, NPJ Genome Med., August 12; 6(1):67, herein incorporated by reference in its entirety. Specifically, the mutation list can be found in Supplementary Data 2 of Gutman et al.

TABLE 5
Genes with silent mutations for each cancer.

| Cancer | | | | |
|---|---|---|---|---|
| (type) | Intron | UTR | Flank | Synonymous |
| BLCA | KMT2C, SET, SEPT9, | PABPC1, FAM46C, | DUX4, MLLT4, U2, | MUC4, AK2, |
| (bladder | PRKAR1A, DCC, | PAX3, BCR, TRIM2, | AK2, RGPD3, | MUC16, CHEK2, |
| cancer) | CNTNAP2, ELF3, | TEC, HLA-A, PAX3, | LARP4, BCR, WT1, | KMT2C, LHFP, |
| | PDE4D, TET2, PARG | LARP4, HMGN2 | MALAT1, CRTC1 | HLA-A, AK2, |
| | | | | CHEK2, WHSC1L1 |
| BRCA | PAX3, TFG, CR1, | TEC, HIP1, CAMTA1, | DUX4, U2, YOD1, | MUC4, AK2, HOXA1, |
| (breast | BCLAF1, CTNNA2, | PAX3, EIF3E, LARP4, | MALAT1, ZNRF3, | MUC16, CLIP1, |
| cancer) | KMT2C, SLC34A2, | HLA-A, TBL1X, | MLLT4, AK2, | USP8, KMT2C, |
| | ZNF521, DDR2, CR1 | PHOX2B, PPP6C | RGPD3, MUC1, SGK1 | CREBBP, TSHR, CHEK2 |
| CESC | CARD11, PAX3, | PABPC1, BCR, CSF1, | RGPD3, BCR, | MUC4, KMT2C, |
| (cervical | SEPT9, RGPD3, | FOXO3, RNF4, | PRDM16, U2, | HLA-A, CHEK2, |
| cancer) | ZNRF3, ACSL3, | SRGAP3, LARP4, | DUX4, NR4A3, | MUC16, AK2, |
| | DDR2, PIK3CB, | TRIM2, RARA, PDGFB | WWTR1, USP6, | RANBP2, TF, |
| | MLLT4, PDGFB | | AK2, DDX6 | NUP98, BRD3 |
| COAD | CNTRL, PARG, TPR, | FAM46C, EIF3E, TEC, | YOD1, U2, DUX4, | RANBP2, AK2, |
| (colon | KMT2C, STAG2, | BCL11A, MLLT11, | ZNRF3, AK2, | MUC4, CLIP1, |
| cancer) | PTPRT, FUS, ROBO2, | BCR, FST, UBR5, | MALAT1, MYH1, | MAP3K13, RANBP2, |
| | KIAA1598, PAX3 | MUC16, TRIM2 | NFATC2, ALK, LCP1 | KMT2C, FANCD2, |
| | | | | MUC16, CHEK2 |
| GBM | EZH2, PARG, CR1, | LARP4, PABPC1, | U2, MLLT4, LARP4, | MUC4, MUC16, TF, |
| (brain | EGFR, ARHGAP26, | FAM46C, BCL11A, | MDS2, AK2, DUX4, | MECOM, KMT2C, |
| cancer) | CSMD3, SEPT9, | CCR7, QKI, CSMD3, | RGPD3, MALAT1, | MAP3K13, EGFR, |
| | DDR2, COL1A1, CBLC | SDHA, FGFR3, MSN | FANCD2, WWP2 | AFF4, AK2, FAT1 |
| HNSC | KMT2C, PARG, SET, | PABPC1, PAX3, | DUX4, MLLT4, AK2, | MUC4, MUC16, |
| (head | PAX3, PAX5, SEPT9, | TBL1X, FAM46C, BCR, | RGPD3, U2, CRTC1, | CLIP1, KMT2C, |
| and neck | CNTNAP2, TET2, | IL7R, CBL, CAMTA1, | MALAT1, DDX6, | CHEK2, HLA-A, |
| cancer) | CARD11, PTPRT | EIF3E, SDHA | MECOM, CRTC1 | PABPC1, BRD3, |
| | | | | MECOM, FANCD2 |
| KIRC | EZH2, FAS, SETBP1, | IL7R, LARP4, EIF3E, | AK2, U2, LARP4, | KMT2C, FAT3, FAT1, |
| (renal | SMARCA4, PTPN6, | SDHA, BCL11A, IL2, | DUX4, MALAT1, | ROBO2, PICALM, |
| cancer) | MLLT4, CSMD3, F5, | TRIM2, BCR, DCT, | RGPD3, ZNRF3, | RANBP2, MUC4, |
| | KMT2C, MUC16 | ERCC2 | YOD1, STK11, | BRCA2, PARG, |
| | | | MLLT4 | ARID2 |
| KIRP | SET, PCM1, | GNAS, PHOX2B, | MUC1, U2, DUX4, | MUC16, MUC4, |
| (renal | MUC16, PDE4D, | CAMTA1, FAM46C, | AK2, RGPD3, | KMT2C, CHEK2, |
| cancer) | RGPD3, KMT2C, | PHOX2B, SRGAP3, | H3F3B, MALAT1, | NUTM2A, PER1, |
| | CSMD3, SEPT9, | CAMTA1, PLAG1, | BCR, DDX6, MLLT4 | NUP98, HLA-A, |
| | ALB, CNTNAP2 | ARHGEF12, EIF3E | | SDHA, PER1 |
| LGG | PTPRT, RGS7, | PAX3, PABPC1, | DUX4, U2, AK2, | MUC4, FAM135B, |
| (brain | CSMD3, PARG, | FAM46C, HIP1, | MLLT4, MALAT1, | CSMD3, DCC, |
| cancer) | CR1, LRP1B, | TRIM2, EBF1, BCR, | RGPD3, BCR, | HOXA1, NSD1, |
| | KMT2C, SET, | RSPO3, SRGAP3, | GATA3, RDM1, RGS7 | MUC16, CHEK2, |
| | SEPT9, PBRM1 | RNF4 | | FAT4, CTNND2 |
| LIHC | SET, SDHA, SEPT9, | GNAS, FAM46C, | DUX4, MUC1, | MUC4, MUC16, |
| (liver | ALB, CDC73, | PHOX2B, PRRX1, | YOD1, TERT, | RANBP2, FCGR2B, |
| cancer) | DDX3X, MUC16, | EIF3E, CAMTA1, | MALAT1, MLLT4, | SETBP1, TAL1, |
| | PARG, HLA-A, | SRGAP3, SDHA, | CRTC1, AK2, U2, | KMT2C, MAP3K13, |
| | PTPRC | MLLT11, PABPC1 | ZNRF3 | AK2, CHEK2 |
| LUAD | KMT2C, SET, | PABPC1, FAM46C, | DUX4, MLLT4, U2, | MUC4, MUC16, |
| (lung | LRP1B, SEPT9, | PAX3, BCR, SDHA, | AK2, RGPD3, | NUP98, AK2, |
| cancer) | CSMD3, FHIT, | RSPO3, EIF3E, | MALAT1, SP1, | KMT2C, FAT3, |
| | PDE4D, PARG, | PIK3R1, HMGN2, | SGK1, CDKN1A, BCR | RNF43, HLA-A, |
| | CDH10, CNTNAP2 | HLA-A | | CHEK2, CSMD3 |
| LUSC | PARG, SET, CDH10, | FAM46C, PABPC1, | DUX4, MLLT4, U2, | MUC4, CDH10, |
| (lung | CSMD3, KMT2C, | CAMTA1, EIF3E, | RGPD3, AK2, | MUC16, KMT2C, |
| cancer) | PAX3, FANCD2, | SDHA, FGFR3, | MALAT1, ZNRF3, | PABPC1, CSMD3, |
| | CTNND2, SEPT9, | CDH10, HLA-A, | CRTC1, LARP4B, | CHEK2, AK2, |
| | FAM135B | PHOX2B, FST | CDKN1A | MECOM, FAT1 |
| OV | ARHGAP26, DDX6, | PAX3, FAM46C, | AK2, MALAT1, U2, | MUC4, HOXA1, |
| (ovarian | ANK1, NCOA1, | SDHA, TEC, PABPC1, | MLLT4, DUX4, | AMER1, MUC16, |
| cancer) | MYH11, SF3B1, | CAMTA1, QKI, | MDS2, MUC16, | CLIP1, RANBP2, |
| | CASP8, HLA-A, | MLLT11, MAF, JAK3 | PRDM16, RGPD3, | KMT2C, SDHA, |
| | MUC16, NACA | | STK11 | CHD4, STIL |
| PRAD | KMT2C, CNTNAP2, | FAM46C, PAX3, | DUX4, LARP4, | MUC4, KMT2C, |
| (prostate | TG, CR1, SET, | PABPC1, PPP6C, TEC, | MLLT4, U2, | CHEK2, UBR5, |
| cancer) | NTRK3, PARG, | SDHA, BCR, HIP1, | MALAT1, AK2, | MUC16, APOB, |
| | PTPRT, RGPD3, | BCL2, EPHA3 | RGPD3, WWP2, | TRRAP, PTPRT, |
| | LRP1B | | SDHC, PRDM16 | CNTNAP2, CSMD3 |
| SARC | CARD11, PAX3, | BCR, SRGAP3, | NR4A3, RGPD3, | MUC4, RANBP2, |
| (bone | HLA-A, RGPD3, | PABPC1, CSF1, RNF4, | BCR, PRDM16, SP1, | KMT2C, CHEK2, AK2, |
| and soft | ZNRF3, CR1, CTCF, | FOXO3, NF1, TRIM2, | DUX4, USP6, PAX8, | NUP98, HLA-A, |
| tissue | SEPT9, PARG, | PLAG1, ZNRF3 | MLLT4, DDX6 | MECOM, PABPC1, |
| cancer) | CTNNA2 | | | MUC16 |
| SKCM | PTPRT, MYH1, | PABPC1, CAMTA1, | DUX4, MALAT1, | MYH1, MUC4, |
| (skin | PARG, C6, KMT2C, | TRIM2, CD209, SDHD, | PMS2, FAT3, | GRIN2A, TRRAP, |
| cancer) | C3, MUC16, SET, | PAX3, SDHA, GRM3, | MLLT4, ZNF384, | MUC16, PTPRT, RP1, |
| | LRP1, SLC34A2 | TEC, LARP4 | CDKN1A, AK2, | KMT2C, NUP98, |
| | | | TFEB, WNK2 | APOB |
| STAD | SET, CR1, TSC2, | TRIM24, FAM46C, | MALAT1, MLLT4, | MUC4, CLIP1, |
| (stomach | APOB, KMT2C, | PABPC1, FOXP1, | DUX4, AK2, U2, | MUC16, COL3A1, |
| cancer) | SEPT9, PARG, | PAX3, SDHA, | LARP4, TNFRSF14, | AK2, KMT2C, SDHA, |
| | EP300, LRP1B, | HMGN2, RSPO3, TEC, | RGPD3, BCL11A, | CHEK2, BCLAF1, |
| | PAX3 | HLA-A | KLK2 | FAT1 |
| THCA | RGS7, SET, LRP1B, | PABPC1, PAX3, | DUX4, U2, MLLT4, | MUC4, CSMD3, AK2, |
| (thyroid | CSMD3, SEPT9, | CAMTA1, FAM46C, | AK2, RGPD3, | RANBP2, MYH1, MGA, |
| cancer) | CNTNAP2, PDE4D, | TRIM2, TBL1X, | ZNF384, CRTC1, | MUC16, HLA-A, |
| | PAX3, PARG, ROBO2 | BCORL1, SDHA, | SP1, ARHGEF1, | HOXA1, KMT2C |
| | | CSMD3, KDM5A | DDX6 | |
| UCEC | PARG, TFG, CR1, | TBL1X, BCL11B, BCR, | AK2, SP1, DUX4, | MUC4, AK2, HOXA1, |
| (uterine | PAX3, KDM6A, | CCND2, ABI1, | YOD1, U2, RGPD3, | CLIP1, CHD4, KMT2C, |
| cancer) | SETBP1, BCLAF1, | PABPC1, PAX3, EXT1, | MLLT4, PPP2R1A, | USP8, HLA-A, MUC16, |
| | SLC34A2, KMT2C, | RNF4, FBLN2 | FGFR2, MECOM | RANBP2 |
| | RGPD3 | | | |

### Example 5: Synonymous, Non-Silent and Intronic Mutations Affect a Gene's Predictive Power on Cancer Type Classification in a Positively Correlated Manner

To assess whether some mutation types lead to similar consequences, every cancer type was separately examined. It was assumed that if two different mutation types have similar effects on a gene, then the predictive power of that gene for a specific cancer type would be similar when mutated by either one of them. Therefore, the gene's importance in both models should be similar as well. Inferring to all genes, the gene importance ranking of both models should be correlated.

For every cancer type, a Spearman correlation was performed between every pair of gene ranking lists obtained from the five single-mutation-type models (see Methods). The correlation coefficients were then averaged across all cancer types. The results (FIG. 6) indicate a significant 0.4 correlation between the gene ranking lists of the non-silent and synonymous models, a 0.32 correlation between the lists of the non-silent and intron models and a correlation of 0.3 between the lists of the synonymous and intron models. These three correlations obtained a p-value smaller than 8.5*10\hat{ }(−9). Correlations between all other pairs of models were neither high nor significant. A possible reason for these results is a common mechanism shared by the different mutation types. For example, both synonymous and non-silent mutations may affect co-translational folding, and both synonymous and intronic mutations may influence splicing. Thus, it is conceivable that these mutations could have similar consequences over the gene's expression or functionality.

### Example 6: Combining Both Silent and Non-Silent Features Enables the Detection of Gene Ontology Terms that are not Detected by Non-Silent Features Alone

Enrichment analysis was performed in order to examine whether genes that were considered important by the models are related to specific biological functions and processes. The affiliation of these genes to biological pathways could illuminate their contribution to the development and progression of the disease. The GOrilla and REVIGO tools were used to find non-redundant Gene Ontology terms (GO terms) that are enriched for any of the 19 cancer types. To find the terms, a gene ranking list was used as input for the GOrilla tool (see Methods). As demonstrated in FIG. 5 and FIG. 6, different mutation types dramatically change the predictive power of genes and thus inputting gene rankings of the different models could illuminate different biological pathways.

FIG. 7 lists the GO terms that were enriched for the 19 cancer types when using the gene rankings from the all-features models. Examining these results, it can be seen that most GO terms that are repeatedly enriched across cancer types are related to DNA-protein bindings, to protein-protein bindings and to phosphorylation. As expected, these terms are associated with various regulation mechanisms of the gene expression process, such as transcription (interactions between transcription factors and RNA Polymerase, histone phosphorylation) or translation (attachment of ribosomes to the DNA sequence).

As most research today encompasses mainly non-silent mutations, it is interesting to test whether the GO terms that were detected with the all-features gene rankings are also detected with gene rankings obtained from non-silent models. FIG. 8 depicts the number of cancer types for which a GO term was found significantly enriched when using the gene rankings from both models. It can be seen that most GO terms detected by the all-features models across various cancer types are considerably less detected by the non-silent models. That is to say, adding silent features to non-silent features caused the gene ranking to encompass a broader biological significance and thus led to a more comprehensive detection of GO terms. Nonetheless, widening the prism involves a trade-off, 10 GO terms that were found significant by the non-silent model were missed by the all-features model (in fact, eight of them were missed by all other models, making them unique to the non-silent model). Among these terms are “endothelial cell migration” which is related to angiogenesis (a known cancer hallmark), “negative regulation of morphogenesis of an epithelium” which is indeed affected in carcinoma development and “regulation of canonical Wnt signaling pathway” which is known to be profoundly related to cell tumorigenesis. These terms were found significant only by the non-silent model and neither they, nor semantically similar terms, were detected by any other model. Even though the all-features model missed these 10 terms, it did detect the other 21 terms that were found significant by the non-silent model, meaning that the majority of the information was preserved. Additionally, it detected 90 other significant GO terms that were not detected by the non-silent model. These include terms related to histone modifications (“histone binding”, “histone methyltransferase activity”, “histone acetyltransferase activity”), terms related to phosphorylation (“transmembrane receptor protein phosphatase activity”, “transmembrane receptor protein kinase activity”) and terms related to the binding of nucleic acids (“ATP binding”, “GDP binding”, “GTPase activator activity”). These biological functions and processes are known to have implications on tumorigenesis in various ways and none of them (or terms with similar semantic meanings) were detected by the non-silent model. Pathway enrichment analysis was also performed using REACTOME (see Methods) and the results indicate that the all-features highly ranked genes are associated with multiple pathways related to regulation of DNA damage. Pathways such as “Cell cycle checkpoints” (and specifically “G1/S DNA Damage Checkpoints”, “G2/M DNA damage checkpoint” and “p53-Dependent G1 DNA Damage Response”), “DNA double-strand break repair”, “SUMOylation of DNA damage response and repair proteins” and “TP53 Regulates Transcription of DNA Repair Genes” were enriched. These pathways, or any semantically similar pathways were not found enriched in the highly ranked genes of the non-silent models and are known to be profoundly related to tumorigenesis. This further demonstrates the contribution of silent mutations to tumorigenesis and highlights the need to combine them in cancer research.

Examining the single-feature-type silent models, more GO terms are detected that were unique to a specific model. For example, the term “poly(A) binding” was found significant only by the UTR model. This may suggest that poly(A) binding genes tend to undergo regulation and thus also cancer evolution through mutations in their 3′UTR which affect regulation via the changes in the poly(A) tail. The poly(A) tail is related to mRNA stability and translation regulation and alternative polyadenylation processes are known to be related to tumorigenesis. Another example for a term that is unique for a specific model only is “O-glycan processing” which was found significant only by the synonymous model. The O-glycans are oligosaccharides that are a major component of mucins. The mucins function as a protective layer of the epithelium and changes in their O-glycans are related to tumorigenesis.

The intron model also detected many significant GO terms for the various cancer types (80), only three of which (“cell adhesion”, “biological adhesion” and “integral component of plasma membrane”) are common with the non-silent model. Exactly half of the terms (40) were also detected by the all-features model. To conclude, there is a trade-off in examining gene rankings obtained from single-feature-type models and models that combine several feature types. The all-features model allows for a broader view of biological pathways but also misses terms that are highly specific of a certain mutation type. Though examples are given here for UTRs, introns and synonymous models, there were also GO terms found in the flanking regions that did not appear when the combined model was used. However, this analysis strongly indicates that searching for biological significance by only analyzing non-silent mutations is insufficient.

When examining the results depicted in FIG. 8, one must consider the uneven number of features in both models; The all-features models have almost seven times as many features as the non-silent models. Because the gene ranking is derived from the feature ranking it is bound to have some effect over the enrichment results. However, it is not the only determinant; if the silent features were unimportant for the model, adding them (even many of them) would not cause such a difference in the enrichment results. As the rank of a gene is derived from the rank of its most important feature (see Methods), unimportant silent features would have made a small impact on the gene ranking, leading to similar gene rankings of the all-features and non-silent models and thus to similar enrichment results. The fact that many more GO terms were found enriched by the all-features models demonstrates once again the importance of the silent features and the importance of examining the whole picture.

### Example 7: All Silent-Features Models Outperformed the Null Model in Predicting Survival Probabilities for More than 10 Years after an Initial Cancer Diagnosis

One purpose of this analysis was to assess whether the survival probabilities of patients could be estimated solely based on their silent mutations, and to compare the estimations of the silent-features models to the estimations of the non-silent and all-features models. Similar to the cancer type classification task, no additional information, such as patient's age, sex, race or treatment history was used. In this analysis, patients across all 33 cancer types were included and a Random Survival Forest (RSF) algorithm was utilized (see Methods). Due to the high computational requirements of the algorithm, only a subset of the features was chosen from each of the six initial datasets. The models were trained to predict patients' survival probability at any time after an initial cancer diagnosis. Then, the models were used to estimate the survival probabilities of patients at 10 different time points. The estimations were evaluated using the Area Under the Curve (AUC) score and the results are presented in the following section.

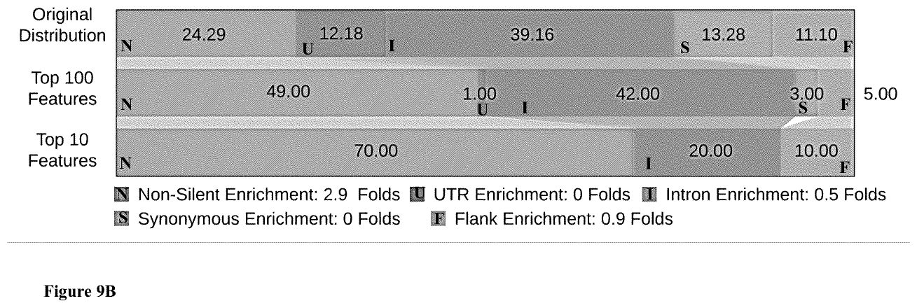

All the silent-features models outperformed the null model for more than 10 years after the initial diagnosis (FIG. 9A). Additionally, the all-features model achieved the highest AUC score for more than nine years (3,500 days) after the diagnosis. This demonstrates that the addition of silent features to non-silent features is superior to the use of non-silent features alone for survivability prediction.

### Example 8: Silent Features Comprise 30% of the 10 Most Predictive Features for Survival Estimation

Reviewing the feature importance ranking produced by the all-features model for survival estimation, silent features comprised more than half of the top ranked 100 features and a third of the top ranked 10 features (FIG. 9B). Table 6 holds the 10 most predictive features for survival estimation. Note that due to technical reasons (see Methods) all patients are treated as a single cohort for the survival estimation (the cancer type of each patient is not considered by the model, only the patients' genomic features and vital status at the last examination). If one were to perform a separate survival analysis for each cancer type as was done in the classification task, it is probable that the number of highly ranked silent mutations would vary significantly among the cancer types as seen in the previous task (Tables 2 and 3). However, the fact that three of the 10 features that are most predictive of the survivability of the entire cohort are silent (even though thousands of non-silent features were available for the model's usage), is another indicator of the strong predictive ability of silent mutations.

TABLE 6
The top 10 ranked features for estimating patients' survival probability.
For each feature, the table holds its name, mutation type, its importance
ranking, the gene to which it is related to and the gene's product
description. The ranking was obtained from the all-features model.

| Rank | Feature | Feature Type | Importance | Gene |
|---|---|---|---|---|
| 0 | TP53 Non_Silent | Non_Silent | 0.0142 | TP53 |
| 1 | MUC4 Non_Silent | Non_Silent | 0.0050 | MUC4 |
| 2 | 57466291- | Non_Silent | 0.0049 | GLI1 |
| | 57466292-chr12- | | | |
| | Frame_Shift_Ins- | | | |
| | INS-----G | | | |
| 3 | 143147221-143147222- | Intron | 0.0035 | ARHGAP26 |
| | chr5-Intron- | | | |
| | INS-----C | | | |
| 4 | 140753336- | Non_Silent | 0.0032 | BRAF |
| | 140753336-chr7- | | | |
| | Missense_Mutation- | | | |
| | SNP-A-A-T | | | |
| 5 | NKX2-1 Flank | Flank | 0.0031 | NKX2-1 |
| 6 | 25743660- | Non_Silent | 0.0027 | ASXL2 |
| | 25743660-chr2- | | | |
| | Missense_Mutation- | | | |
| | SNP-T-T-G | | | |
| 7 | EGFR Non_Silent | Non_Silent | 0.0026 | EGFR |
| 8 | 92956452-92956452- | Intron | 0.0026 | CHD2 |
| | chr15-Splice_Region- | | | |
| | SNP-A-A-T | | | |
| 9 | 35457937- | Non_Silent | 0.0026 | FANCE |
| | 35457938-chr6- | | | |
| | Frame_Shift_Ins- | | | |
| | INS-----C | | | |

### Example 9: Other Types of Analysis

As demonstrated by the evaluation of both cancer type and survival time, silent mutations are useful for any cancer objective. That is, any cancer evaluation where non-silent mutations are known to informative, silent mutations are also informative and the combination of both types of mutations is potentially most informative.

Databases of sequencing data from subjects with and without cancer, with known driver mutations, or with known responsiveness to a particular therapeutic treatment are accessed. Mutational datasets are prepared as before and analyzed as before. Trained machine learning models are produced for each analysis to be performed (diagnosis, driver analysis, companion diagnosis/response). The subjects are dived into two sets a training set (70%) and a test set (30%). These sets are randomly generated multiple times in order to increase the accuracy/effectiveness of the model. The trained models are confirmed on other sets of subjects not used for generating and the model and are found to be able to predict the presence of cancer, the mutational driver and/or the responsiveness of subjects.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining a type of cancer in a subject or estimating survival time after diagnosis of a subject, the method comprising:
a. receiving genomic mutation data from said cancer wherein said data comprises mutations that are not exonic non-synonymous mutations;
b. applying a trained machine learning (ML) model to said received genomic mutation data;
thereby determining a type of cancer in a subject or estimating survival time after diagnosis for said subject.

2. The method of claim 1, wherein said data comprises mutations found in said cancer which are absent from healthy tissue of said subject.

3. The method of claim 1, wherein said method is a method of determining cancer type and said ML model was trained on a training set comprising said genomic mutation data from cancer patients with known cancer types and said ML model outputs a classification of said cancer in said subject as one of said known cancer types.

4. The method of claim 1, wherein said method is a method of estimating survival time after diagnosis of said subject and said ML model was trained on a training set comprising said genomic mutation data from cancer patients with known survival times from diagnosis and said ML model outputs an estimated survival time for said subject.

5. The method of claim 3, wherein said training set comprises only mutations that appear in at least two of said cancer patients with known cancer types.

6. The method of claim 1, wherein said mutations are selected from: mutations in 3′ and 5′ untranslated regions (UTRs) of genes, mutations in introns of genes, mutations in regions flanking genes comprising untranscribed sequences within 5 kb of a transcriptional start site of genes, within 5 kb of a transcriptional termination site of genes or both, and exonic synonymous mutations.

7. (canceled)

8. The method of claim 1, wherein said genomic mutation data comprises:
a. all UTR mutations in deep sequencing data from said subject, said cancer patients or both;
b. all intronic mutations in deep sequencing data from said subject, said cancer patients or both;
c. all flanking region mutations in deep sequencing data from said subject, said cancer patients or both;
d. all synonymous exonic mutations in deep sequencing data from said subject, said cancer patients or both; or
e. a combination thereof.

9. The method of claim 1, wherein said genomic mutation data further comprises exonic non-synonymous mutations, said genomic mutation data is from a cancer biopsy or liquid biopsy or both.

10. The method of claim 9, wherein said genomic mutation data comprises all exonic non-synonymous mutations in deep sequencing data from said subject, said cancer patients or both.

11. The method of claim 8, wherein said deep sequencing is whole exome sequencing (WES).

12. The method of claim 11, wherein said genomic mutation data comprises all mutations found in WES data from said subject, said cancer patients or both.

13. The method of claim 1, wherein said cancer is selected from adrenal cancer, bladder cancer, urothelial cancer, breast cancer, cervical cancer, bile duct cancer, colon cancer, lymphoid cancer, esophageal cancer, brain cancer, head and neck cancer, renal cancer, liver cancer, lung cancer, mesodermal cancer, ovarian cancer, pancreatic cancer, endocrine cancer, neuroendocrine cancer, prostate cancer, rectal cancer, skin cancer, bone cancer, soft tissue cancer, stomach cancer, testicular cancer, thyroid cancer, uterine cancer and uveal cancer.

14. The method of claim 13, wherein said genomic mutation data comprises intronic mutations and said cancer is selected from cervical cancer, colon cancer, brain cancer, renal cancer, and liver cancer.

15. The method of claim 13, wherein said genomic mutation data comprises UTR mutations or flanking region mutations and said cancer is selected from cervical cancer, bone cancer and soft tissue cancer.

16. The method of claim 13, wherein said genomic mutation data comprises UTR mutations, intronic mutations, flanking region mutations, exonic synonymous mutations and exonic non-synonymous mutations and said cancer is selected from bladder cancer, urothelial cancer, breast cancer, cervical cancer, colon cancer, brain cancer, renal cancer, liver cancer, lung cancer, ovarian cancer, bone cancer, soft tissue cancer, skin cancer, thyroid cancer and uterine cancer.

17. (canceled)

18. (canceled)

19. The method of claim 1, further comprising administering to said subject a therapeutic agent known to treat said determined cancer type or administering an additional therapeutic treatment to a subject with an expected survival time below a predetermined threshold.

20. (canceled)

21. A method comprising:
training a machine learning (ML) model to determine a type of cancer in a subject or estimate survival time after diagnosis of a subject, on a training set, the method comprising:
i. receiving genomic data; and
ii. extracting from said received genomic data mutations, wherein said mutations are not exonic non-synonymous mutations;
wherein said training set is generated by labeling said mutations as coming from a cancer of a specific type or from a subject that survived for a specific amount of time after diagnosis and combining a plurality of mutations and their labels together to form said training set, wherein said plurality comprises labels of cancers from at least two cancer types or labels from subjects that survived for different amounts of time.

22. The method of claim 21, further comprising at an inference step applying said trained ML model to genomic mutation data received from a cancer wherein said received genomic data comprises mutations that are not exonic non-synonymous mutations and outputting a determined type of cancer or an estimated survival time.

23. (canceled)

24. A method of evaluating a cancer, the method comprising receiving a sample comprising DNA from said cancer and detecting in said DNA a silent mutation in a gene selected from those provided in Table 5, thereby evaluating a cancer.

25. The method of claim 24, wherein at least one of:
a. said cancer is selected from a cancer type provided in Table 5 and wherein said gene is selected from those whose mutation was observed in said cancer type;
b. said evaluating comprises determining a driver gene or driver mutation in said cancer; and
c. said method further comprises administering to a subject that provided said sample an anticancer therapy that targets a driver gene determined in said cancer, another gene in a biological pathway comprising said determined driver gene or said driver mutation.

26. (canceled)

27. (canceled)
